(19) United States
(12) Patent Application Publication
(10) Pub. No.: US 2026/0347444 A1
(43) Pub. Date: Nov. 5, 2026

(54) PROTEOLYSIS TARGETING ANTIBODIES AND METHODS OF USE THEREOF

(71) Applicant: YALE UNIVERSITY, New Haven, CT (US)

(72) Inventors: Peter GLAZER, New Haven, CT (US); Elias QUIJANO, New Haven, CT (US); Audrey TURCHICK, New Haven, CT (US)

(21) Appl. No.: 19/110,059

(22) PCT Filed: Sep. 11, 2023
(86) PCT No.: PCT/US2023/073856

§ 371 Date: Mar. 10, 2025

Related U.S. Application Data
(60) Provisional application No. 63/375,140, filed on Sep. 9, 2022.

(51) Int. Cl. C07K 16/18; A61K 39/00

(52) U.S. Cl. CPC C07K 16/18; A61K 2039/54; C07K 2317/24; C07K 2317/31; C07K 2317/524; C07K 2317/526; C07K 2317/565; C07K 2317/77

(57) ABSTRACT

Antigen binding molecules for targeted proteolysis are provided. The molecules typically include (1) a TRIM21 binding feature, (2) the cell-penetrating feature of a cell-penetrating antibody, and (3) an antigen binding feature that binds to a target protein. The TRIM21 binding feature typically includes a polypeptide sequence sufficient to bind to TRIM21 and is effective to direct the target protein along the TRIM21 degradation pathway. The cell-penetrating feature of a cell-penetrating antibody is typically one that enhances delivery of the molecule across cell membranes. The antigen binding feature binds to a target protein and mediates binding of the molecule to the target protein. In some embodiments, the antigen binding molecule is a multispecific antibody, such as a bi- or tri-specific antibody. Composition including an effective amount of the antigen binding molecules and methods of use thereof are also provided.

### CROSS-REFERENCE TO RELATED APPLICATIONS

This application claims the benefit of and priority to U.S. Provisional Application No. 63/375,140 filed Sep. 9, 2022, which is hereby incorporated by reference in its entirety.

### STATEMENT REGARDING FEDERALLY SPONSORED RESEARCH OR DEVELOPMENT

This invention was made with government support under R35 CA197574 awarded by the National Institutes of Health. The government has certain rights in the invention.

### REFERENCE TO SEQUENCE LISTING

The Sequence Listing submitted as a text file named “YU_8465_PCT_ST26.xml”, created on Sep. 11, 2023, and having a size of 131,184 bytes is hereby incorporated by reference pursuant to 37 C.F.R. § 1.52(e)(5).

### FIELD OF THE INVENTION

The field of the invention is generally in the field of targeted degradation of protein.

### BACKGROUND OF THE INVENTION

Methods for the targeted disruption of protein function have revolutionized science and greatly expedited the systematic characterization of genes. Two main approaches are currently used to disrupt protein function: DNA knockout and RNA interference, which act at the genome and mRNA level, respectively. An antibody-based technique to degrade endogenous proteins acutely in mammalian cells without prior modification of the genome or mRNA has been developed, however, “Trim-Away” typically utilizes to transfection assistance such as electroporation to facilitate entry of the compositions into cells, limiting its use in the clinical setting (Clift, et al., Cell. 2017; 171 (7): 1692-1706.e18. doi: 10.1016/j.cell.2017.10.033; Clift, Nat Protoc 13, 2149-2175 (2018). doi.org/10.1038/s41596-018-0028-3; Zeng, Nat Struct Mol Biol 28, 278-289 (2021). doi.org/10.1038/s41594-021-00560-2).

Thus, there remains as a need for improved compositions and methods in this field.

It is object of the invention to provide improved compositions and methods for targeted proteolysis.

### SUMMARY OF THE INVENTION

Antigen binding molecules for targeted proteolysis and methods of use thereof are provided. The molecules typically include (1) a TRIM21 binding feature, (2) the cell-penetrating feature of a cell-penetrating antibody, and (3) an antigen binding feature that binds to a target protein.

The TRIM21 binding feature typically includes a polypeptide sequence sufficient to bind to TRIM21 and is effective to direct the target protein along the TRIM21 degradation pathway when bound by one or more antigen binding molecules. A preferred TRIM21 binding feature includes the interface of the CH2 and CH3 domains of an immunoglobulin constant region, more preferably the CH2 and CH3 domains of an immunoglobulin constant region (e.g., Fc region), optionally wherein the immunoglobulin is an IgG, optionally a human IgG, such as IgG1.

The cell-penetrating feature of a cell-penetrating antibody is typically one that enhances delivery of the antigen binding molecule across cell membranes. The cell-penetrating feature can be the cell-penetrating domain of an autoantibody such as 3E10 or 5C6, or a cell-penetrating variant or humanized form thereof. In some embodiments, the antigen binding molecule includes the complementary determining regions (CDR), and optionally the heavy and light chain variable regions of the cell-penetrating antibody (e.g., 3E10, 5C6, or a variant or humanized form thereof) regardless of whether or not such regions are important for cell penetration. In some embodiments, the antigen binding molecule includes the Fc region of cell-penetrating antibody, regardless of whether or not such region is important for cell penetration.

The antigen binding feature binds to a target protein and mediates binding of the molecule to the target protein. Some molecules feature two or more antigen binding features that bind to different epitopes on the same target protein, different epitopes on different proteins, and or a combination thereof. The antigen binding feature(s) can be traditional VH and VL domains, a single chain antibody, a single chain variable fragments (scFv), di-scFv, tri-scFv, diabody, triabody, teratbody, disulfide-linked Fvs (sdFv), Fab′, F(ab′)2, Fv, a single domain antibody fragments (sdAb), nanobody, or VHH. In some embodiments the antigen binding feature is derived from a known antibody(ies) to the target protein. In some embodiments, the antigen binding molecule includes the Fc region of the known antibody.

In some embodiments, the antigen binding molecules include additional sequence(s) or conjugates. An exemplary additional sequence is the amino acid sequence of TRIM21, or a functional fragment or variant thereof. Thus, in some embodiments, the antigen binding molecule is or includes a TRIM21 fusion protein.

In some embodiments, the antigen binding molecule is a multispecific antibody, such as a bi- or tri-specific antibody. In some embodiments, the bispecific and trispecific antibody is a bispecific or trispecific IgG, IgG-scFv, IgG-dAb, scFv-Fc-scFv, knob-in-hole (KIH)-IgG, κλ-body, KIH0Fc-Fab/scFv, tandom scFv, KIH trispecific, bispecific Fc fusion (N- or C-terminal, with or without KIH).

Compositions including an effective amount of the antigen binding molecules are also provided. In some embodiments, the compositions include an effective amount of two or more different antigen binding molecules. In some embodiments, the compositions include two or more different antigen binding molecules that target the same protein, two or more different antigen binding molecules that target different proteins, or a combination thereof.

Methods of use are also provided and include, but are not limited to, targeted degradation of one or more target proteins. Such methods typically include contacting cells with an effective amount of antigen binding molecules. The contacting can occur in vitro, ex vivo, or in vivo in a subject in need thereof. In some embodiments, the compositions are administered by injection or infusion. In some embodiments, the subject has a disease or disorder, such as one causes by overexpression and/or aberrant expression of the target protein and/or expression of a mutant thereof. In such embodiments, the composition can be administered in an effective amount to treat the disease or disorder.

### BRIEF DESCRIPTION OF THE DRAWINGS

FIGS. 1A-1C are autoradiograms of western blots showing the effect of 3E10 treatment on RAD51 in HCC38 human breast cells (FIG. 1A), YUGASP human melanoma cells (FIG. 1B), and U2OS human osteosarcoma cells (FIG. 1C).

FIG. 2 is an illustration of an exemplary chimeric antibody having a Fab portion containing cell-penetrating feature and the other Fab arm consists of a protein of interest binding feature. The Fc portion contains a TRIM21 binding feature.

FIGS. 3A-3B demonstrate proteosome-mediated degradation of bispecific antibody. FIG. 3A is an image showing steps where B16 cells are treated with cell-penetrating 3E10/GFP bispecific Abs in the presence or the absence of proteosome inhibitor (MG132). FIG. 3B is an image of a Western blot showing the levels of the bispecific antibody in the absence or the presence of proteosome inhibitor MG132.

FIGS. 4A-4C illustrate degradation of nuclear green fluorescent proteins (GFP) after treatment with a cell-penetrating 3E10/GFP bispecific antibody. FIG. 4A show images depicting the levels of the fluorescence generated by control (no antibody) the presence of GFP following incubation with bispecific antibody. FIGS. 4B and 4C are a pair of plots quantifying the amounts of GFP following incubation with control (no antibody, FIG. 4B) and the bispecific antibody (FIG. 4C).

### DETAILED DESCRIPTION OF THE INVENTION

### I. Definitions

As used herein, the term “single chain Fv” or “scFv” as used herein means a single chain variable fragment that includes a light chain variable region (VL) and a heavy chain variable region (VH) in a single polypeptide chain joined by a linker which enables the scFv to form the desired structure for antigen binding (i.e., for the VH and VL of the single polypeptide chain to associate with one another to form a Fv). The VL and VH regions may be derived from the parent antibody or may be chemically or recombinantly synthesized.

As used herein, the term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917).

As used herein, the term “Framework Region” or “FR” residues are those variable domain residues other than the hypervariable region residues as herein defined.

As used herein, the term “antibody” refers to natural or synthetic antibodies that bind a target antigen. The term includes polyclonal and monoclonal antibodies. In addition to intact immunoglobulin molecules, also included in the term “antibodies” are binding proteins, fragments, and polymers of those immunoglobulin molecules, and human or humanized versions of immunoglobulin molecules that bind the target antigen.

As used herein, the term “cell-penetrating antibody” refers to an immunoglobulin protein, fragment, variant thereof, or fusion protein based thereon that is transported into the cytoplasm and/or nucleus of living mammalian cells. A “cell-penetrating anti-DNA antibody” specifically binds DNA (e.g., single-stranded and/or double-stranded DNA). In some embodiments, the antibody is transported into the cytoplasm of the cells without the aid of a carrier or conjugate. In other embodiments, the antibody is conjugated to a cell-penetrating moiety, such as a cell penetrating peptide. In some embodiments, the cell-penetrating antibody is transported in the nucleus with or without a carrier or conjugate.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within +2 is preferred, those within +1 are particularly preferred, and those within +0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

As used herein, the term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

As used herein, the term “specifically binds” refers to the binding of an antibody to its cognate antigen (for example, DNA) while not significantly binding to other antigens. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

As used herein, the term “monoclonal antibody” or “MAb” refers to an antibody obtained from a substantially homogeneous population of antibodies, i.e., the individual antibodies within the population are identical except for possible naturally occurring mutations that may be present in a small subset of the antibody molecules.

As used herein, the term “subject” means any individual who is the target of administration. The subject can be a vertebrate, for example, a mammal. Thus, the subject can be a human. The term does not denote a particular age or sex.

As used herein, the term “effective amount” means that the amount of the composition used is of sufficient quantity to ameliorate one or more causes or symptoms of a disease or disorder. Such amelioration only requires a reduction or alteration, not necessarily elimination. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “pharmaceutically acceptable” refers to a material that is not biologically or otherwise undesirable, i.e., the material may be administered to a subject without causing any undesirable biological effects or interacting in a deleterious manner with any of the other components of the pharmaceutical composition in which it is contained.

As used herein, the term “carrier” or “excipient” refers to an organic or inorganic ingredient, natural or synthetic inactive ingredient in a formulation, with which one or more active ingredients are combined. The carrier or excipient would naturally be selected to minimize any degradation of the active ingredient and to minimize any adverse side effects in the subject, as would be well known to one of skill in the art.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, “targeting moiety” is a substance which can direct a particle or molecule to a receptor site on a selected cell or tissue type, can serve as an attachment molecule, or serve to couple or attach another molecule. As used herein, “direct” refers to causing a molecule to preferentially attach to a selected cell or tissue type. This can be used to direct cellular materials, molecules, or drugs, as discussed below.

As used herein, the term “inhibit” or “reduce” means to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, a “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from a nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid sequence, and then expressing the nucleic acid in an appropriate host cell under conditions in which the fusion protein is produced.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

As used herein, “optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Every compound disclosed herein is intended to be and should be considered to be specifically disclosed herein. Further, every subgroup that can be identified within this disclosure is intended to be and should be considered to be specifically disclosed herein. As a result, it is specifically contemplated that any compound, or subgroup of compounds can be either specifically included for or excluded from use or included in or excluded from a list of compounds.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of polypeptides are discussed, specifically contemplated is each and every combination and permutation of polypeptides and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### II. Proteolytic Systems

### A. Strategies for Targeted Proteolysis

TRIM21 is a multi-domain protein including an N-terminal RING (SEQ ID NO:127) domain with E3 ubiquitin ligase activity, a B-box domain, a coiled-coil dimerization domain and a C-terminal PRYSPRY (SEQ ID NO: 125) domain. The domain architecture is conserved within the TRIM protein family and it is the C-terminal PRYSPRY (SEQ ID NO:125) domain that contains the antibody binding site and thus dictates function. The TRIM21 PRYSPRY (SEQ ID NO:125) domain is a globular fold comprising a β-sandwich of two anti-parallel β-sheets connected by flexible loops, which are sub-divided into PRY and SPRY (SEQ ID NO:126) elements. In solution, TRIM21 exists as a homodimer and forms a stable 1:1 complex with antibody, in which the two PRYSPRY (SEQ ID NO: 125) domains bind symmetrically to the Fc.

It has been discovered that the cell-penetrating domain of cell penetrating antibodies can be used to enhance delivery of other binding proteins across cell membranes and enhance induce degradation of intracellular target proteins through the TRIM21 pathway. Disclosed herein are chimeric antigen binding constructs that penetrate cells and bind to target proteins mediating their destruction through the TRIM21 pathway and the cellular protein degradation machinery.

Typically, the constructs include (1) a TRIM21 binding feature, (2) the cell-penetrating feature of a cell-penetrating antibody, and (3) an antigen binding feature that binds to a target protein. It is believed this system will be effective in rapidly degrading the target protein, thus minimizing the risk that phenotypes are compensated and that secondary, non-specific defects accumulate over time. As discussed in more detail below, because the system utilizes antigen binding domains, it can be applied to a wide range of target proteins using currently available antibody sequences.

### B. Design of Antigen Binding Molecules for Targeted Proteolysis

The disclosed antigen binding molecules for targeted proteolysis typically include three core features: (1) a TRIM21 binding feature, (2) the cell-penetrating feature of a cell-penetrating antibody, and (3) an antigen binding feature that binds to a target protein. Each of these features are discussed in more detail below. In some embodiments, the three features are derived from three different molecules, while in other embodiments, two or more features are derived from a single source. For example, in some embodiments, features (1) and (2) are derived from a cell-penetrating antibody, while (3) is derived from a heterologous molecule. Similarly, in some embodiments, features (1) and (3) are derived from the same source, and (2) is derived from a heterologous cell-penetrating antibody molecule. Likewise, the molecules can include one or more additional features or domains.

As discussed in more detail below, the molecules can be assembled in a myriad of different ways and can form of a variety of traditional and non-traditional antibody and antigen binding structures.

### 1. TRIM21 Binding Domain

The TRIM21 binding feature most typically includes an Fc domain capable of being bound by TRIM21 and drawn into its proteolytic pathway.

With respect to antibodies, the variable region (V region) generally refers to a region that is present in an amino acid sequence at the N-terminal side of an immunoglobulin and is rich in diversity. Because a part other than the variable region has a structure with less diversity, it is called a constant region (C region). The respective variable regions of the heavy chain and the light chain are associated to form an antigen binding site and determine the binding property of the antibody to the antigen.

The antigen binding site is a site that recognizes and binds to an antigen in an antibody, and refers to a site that forms a complementary conformation with an antigenic determinant (epitope). At the antigen binding site, a strong intermolecular interaction occurs between the antigenic determinants. The antigen binding site is constituted by VH and VL including at least three complementarity determining regions (CDRs). In the case of a human antibody, VH and VL each include three CDRs. These CDRs are referred to as CDR1, CDR2, and CDR3, respectively, in order from the N-terminal side.

In the constant region, the heavy chain constant region and the light chain constant region are denoted as CH and CL, respectively. The CH is classified into an alpha chain, a delta chain, an epsilon chain, a gamma chain, and a mu chain which are subclasses of the heavy chain. The CH is constituted by a CH1 domain, a hinge domain, a CH2 domain, and a CH3 domain arranged in order from the N-terminal side, and the CH2 domain and the CH3 domain together are called an Fc region. On the other hand, the CL is classified into two subclasses called a C lamda chain and a C kappa chain. The CH2 and CH3 domains together are called an Fc region.

In the case of human IgG, in the heavy chain, a variable region corresponds to the amino acid sequence at positions 1 to 117 numbered according to the EU index of Kabat et al. (Kabat et al., Sequences of proteins of immunological interest, 1991 Fifth edition), and a constant region corresponds to the amino acid sequence downstream of position 118. In the light chain of a human antibody, the amino acid sequence at positions 1 to 107 numbered according to Kabat et al. (Kabat numbering) corresponds to a variable region, and the amino acid sequence downstream of position 108 corresponds to a constant region.

In the case of human IgG, CH1 refers to a region having the amino acid sequence at positions 118 to 215 indicated by the EU index. Similarly, CH2 refers to a region having the amino acid sequence at positions 231 to 340 indicated by the EU index of Kabat et al., and CH3 refers to a region having the amino acid sequence at positions 341 to 447 indicated by the EU index of Kabat et al. Between CH1 and CH2, an amino acid region rich in flexibility called a hinge region (hereinafter sometimes referred to as a hinge) is present. The hinge region refers to a region having the amino acid sequence at positions 216 to 230 indicated by the EU index of Kabat et al.

The CL refers to a region having the amino acid sequence at positions 108 to 214 indicated by Kabat numbering in the case of the chain of a human antibody, and refers to a region having the amino acid sequence at positions 108 to 215 in the case of the lamda chain.

The features of antibodies important for interacting with TRIM21 have been characterized previously. See, e.g., Foss, et al., “TRIM21—From Intracellular Immunity to Therapy”, Front. Immunol., 10:2049, (2019), doi.org/10.3389/fimmu.2019.02049, which is specifically incorporated by reference herein in its entirety, and references cited therein. Direct binding between antibodies and TRIM21 has been demonstrated and its affinity and mechanism of binding dissected by site-directed mutagenesis and binding studies combined with solving the crystal structure of the human TRIM21 PRYSPRY (SEQ ID NO:125) domain in complex with a human IgG1 Fc fragment.

The structure confirmed that two PRYSPRY (SEQ ID NO:125) domains bind to each side of the homodimeric Fc. The TRIM21 binding site is located at the CH2-CH3 interface of the Fc. This is distal from the binding site for the classical Fcγ receptors and complement factor C1q, but overlaps with that of the neonatal Fc receptor (FcRn), as well as viral and bacterial defense proteins. The TRIM21-IgG interaction is largely pH-independent and unaffected by removal of the bi-antennary N-glycan structure attached to N297 in the Fc CH2 domain. It is, however, sensitive to high salt concentrations.

The core TRIM21-IgG1 interaction is formed between a protruding loop encompassing residues 429-436 in the Fc CH3 domain and a deep binding pocket formed on the surface of the PRYSPRY (SEQ ID NO:125) domain. The apex residues H433, N434, H435, and Y436 (HNHY-motif) of the Fc loop is inserted into the PRYSPRY (SEQ ID NO:125) binding pocket. The residues form a hydrogen bond network with the base of the pocket that is protected from solvent by a shield of hydrophobic side chains. Key interacting residues in the PRYSPRY (SEQ ID NO:125) domain include D355, W381, W383, D452, F450, and W299.

The binding affinity between human IgG1 and the recombinant human PRYSPRY (SEQ ID NO:125) domain has been measured to be in the range of 150-200 nM by both isothermal titration calorimetry and surface plasmon resonance. However, since TRIM21 is homodimeric, its functional affinity upon binding symmetrically to the IgG1 Fc is as low as 0.6 nM as measured by fluorescence anisotropy. This represents an increase of >300-fold compared to monomeric binding, making TRIM21 the highest affinity Fc receptor known in humans. Furthermore, the TRIM21-IgG interaction is highly conserved across species, which is illustrated by the fact that both human and mouse TRIM21 efficiently bind IgG from a range of mammals.

Thus, the disclosed molecules typically include a functional fragment or all of an antibody Fc domain capable to being bound by TRIM21 and drawn into its proteolytic pathway. Such domain typically includes at least the CH2-CH3 interface of the Fc, more preferably the entire CH2-CH3 domains. In some embodiments, the protein targeting structures include one or more of a hinge domain and/or a CH1 domain. Although other isotypes are contemplated, in preferred embodiments, the TRIM21 binding feature is derived from an IgG antibody. The Fc domain can be from a mammal, for example, a mouse, rabbit, goat, horse, chicken, hamster, human, etc. In preferred embodiments, the TRIM21 binding domain is derived from a human IgG, optionally a human IgG1, antibody.

### 2. Cell-Penetrating Feature

The cell-penetrating feature is typically derived from or is part or all of a cell-penetrating antibody. Select lupus anti-DNA autoantibodies can penetrate into live cells. See, e.g., Im et al., “Cell- and nuclear-penetrating anti-dsDNA autoantibodies have multiple arginines in CDR3 of VH and increase cellular level of pERK and Bcl-2 in mesangial cells”, Mol Immunol 67, 377-387 (2015), Jang et al., “A nucleic acid-hydrolyzing antibody penetrates into cells via caveolae-mediated endocytosis, localizes in the cytosol and exhibits cytotoxicity”, Cell Mol. Life Sci., 66:1985-97 (2009), Lee, et al., “Cell-penetrating autoantibody induces caspase-mediated apoptosis through catalytic hydrolysis of DNA”, Bioorg. Med. Chem., 15(5): 2016-23 (2007), Ruiz-Arguelles, et al., “Penetration of anti-DNA antibodies into immature live cells.” J. Autoimmun., 11 (5): 547-56 (1998), Song, et al., “Arginines in the CDR of anti-dsDNA autoantibodies facilitate cell internalization via electrostatic interactions”, Eur. J. Immunol., 38 (11): 3178-90 (2008), Vlahakos, et al., “Murine Monoclonal Anti-DNA Antibodies Penetrate Cells, Bind To Nuclei, And Induce Glomerular Proliferation And Proteinuria In Vivo.” J. Am. Soc. Nephrol. 2 (8): 1345-54 (1992), Yanase, et al., “Receptor-mediated Cellular Entry of Nuclear Localizing Anti-DNA Antibodies via Myosin 1”, J. Clin. Invest., 100:25-31 (1997), Yung, et al., “Anti-DNA antibodies in the pathogenesis of lupus nephritis—The emerging mechanisms”, Autoimmunity Rev., 7 (4): 317-21 (2008), Hansen, et al., Sci Transl Med, 4 (157): 157ra142 (2012), Noble, et al., Cancer Research, 2015; 75 (11): 2285-2291, Noble, et al., Sci Rep-Uk, 4 (2014), and Noble, et al., Nat Rev Rheumatol (2016), each of which is specifically incorporated by reference herein in its entirety.

The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur J Immunol 38, 3178-3190 (2008), Yanase et al., J Clin Invest 100, 25-31 (1997)). 3E10 penetrates cells in an Fc-independent mechanism (as evidenced by the ability of 3E10 fragments lacking an Fc to penetrate cells) but involves presence of the nucleoside transporter ENT2 (Weisbart et al., Sci Rep 5:12022. doi: 10.1038/srep12022. (2015), Zack et al., J Immunol 157, 2082-2088 (1996), Hansen et al., J Biol Chem 282, 20790-20793 (2007)). In some embodiments, the antibodies utilized in the disclosed compositions and methods are ones that penetrates cells in an Fc-dependent mechanism. Thus, as introduced above, in some embodiments, the two features: (1) TRIM21 binding feature, and (2) cell-penetrating antibody feature, are part(s) of the same domain: e.g., an Fc domain from a cell-penetrating antibody. In other embodiments, the antibodies utilized in the disclosed compositions and methods are ones that penetrates cells in an Fc-independent mechanism but involves presence of the nucleoside transporter such as ENT2.

In some embodiments, the cell-penetrating feature is in the context of the cell-penetrating antibody from which it is derived, or a fragment or fusion protein based thereon. Thus, the cell-penetrating feature can include the antigen binding domain of the cell-penetrating antibody from which it is derived.

Although the cell-penetrating molecules are generally referred to herein as “cell-penetrating binding proteins” or “cell-penetrating antibodies,” it will be appreciated that fragments, including antigen-binding fragments, variants, binding proteins and fusion proteins such as scFv, di-scFv, tri-scFv, and other single chain variable fragments, and other cell-penetrating molecules disclosed herein are also expressly provided for use in compositions and methods disclosed herein, provided they include the cell-penetrating domain of the intact antibody.

Cell-penetrating antibodies that can be used in the compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the antibodies typically contain at least the CDRs necessary to maintain antigen binding to the target protein or other antigen, and may also contain one or more copies of the light chain variable region (VL) and heavy chain variable region (VH) of an antibody that binds to the antigen. In some embodiments, each VH and VL includes three CDRs and four FRs, arranged from amino-terminus to carboxy-terminus in the following order: FR1, CDR1, FR2, CDR2, FR3, CDR3, FR4.

In some embodiments, the cell-penetrating domain includes a antigen binding domain (e.g., an anti-DNA antigen binding domain) and is present in the form of a single chain antibody, single chain variable fragments (scFv), di-scFv, tri-scFv, diabody, triabody, teratbody, disulfide-linked Fvs (sdFv), Fab′, F(ab′)₂, Fv, and single domain antibody fragments (sdAb).

The antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable region derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco.

In some embodiments, the cell-penetrating domain is derived from or is part or all of antibody 3E10 or 5C6. A panel of hybridomas, including the 3E10 and 5C6 hybridomas was previously generated from the MRLmpj/lpr lupus mouse model and DNA binding activity was evaluated (Zack, et al., J. Immunol. 154:1987-1994 (1995); Gu, et al., J. Immunol., 161:6999-7006 (1998)). Murine 3E10 can refer to the monoclonal antibody produced by ATCC Accession No. PTA 2439 hybridoma. 5C6 can refer to the monoclonal anti-DNA antibody with nucleolytic activity produced by a hybridoma from MRL/lpr lupus mouse model as described in Noble et al., 2014, Sci Rep 4:5958 doi: 10.1038/srep05958. Non-limiting examples of 3E10 and 5C6 antibody sequences are provided below, and can be used in preparation of the molecules disclosed herein. Thus in some embodiments, the cell-penetrating antibody domain is 3E10 or 5C6 antibody or a variant, fragment, and fusion protein thereof, or a humanized form thereof.

### 3. Target Protein Antigen Binding Feature

The antigen binding feature is a domain to facilitates binding to the target protein and captures it for proteolysis through the TRIM21 pathway. Thus, the antigen binding domain is typically an intact antibody or antibody fragment or fusion protein that binds with high affinity and specificity to the target protein. The antigen binding domain can be in the context of any suitable structure provided the target protein is specifically bound. Antibodies that can be used in the compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen as introduce above. Therefore, the antigen binding domain targeting the target protein typically contains at least the CDRs necessary to maintain antigen binding to the target protein, and may also contain one or more copies of the light chain variable region (VL) and heavy chain variable region (VH) of an antibody that binds to the target protein.

Thus, in some embodiments, the antigen binding feature that targets the target protein is present in the form of a single chain antibody, single chain variable fragments (scFv), di-scFv, tri-scFv, diabody, triabody, teratbody, disulfide-linked Fvs (sdFv), Fab′, F(ab′)₂, Fv, single domain antibody fragments (sdAb), nanobodies, VHH, etc. as discussed above.

### 4. Additional Domains and Conjugates

The molecules can also include additional domains and conjugates that serve to further increase targeted proteolysis, and/or accomplish an alternative or additional function or purpose.

For example, in some embodiments, the molecules include a TRIM21 protein, for a functional fragment or variant thereof that can further enhance proteolysis of the target protein. In some embodiments, the TRIM21 is human TRIM21. Sequences for TRIM21 is known in the art, and can be, for example, MASAARLTMMWEEVTCP ICLDP FVEPVSIECGHSFCQECI SQVGKGGGSVC PVCRQRFLLKNLRPNRQLANMVNNLKEISQEAREGTQGERCAVHGERLHLF CEKDGKALCWVCAQSRKHRDHAMVP LEEAAQEYQEKLQVALGELRRKQELA EKLEVEIAIKRADWKKTVETQKSRIHAEFVQQKNFLVEEEQRQLQELEKDE REQLRILGEKEAKLAQQSQALQELISELDRRCHSSALELLQEVIIVLERSE SWNLKDLDITSPELRSVCHVPGLKKMLRTCAVHITLDPDTANPWLILSEDR RQVRLGDTQQSIPGNEERFDSYPMVLGAQHFHSGKHYWEVDVTGKEAWDLG VCRDSVRRKGHFLLSSKSGFWTIWLWNKQKYEAGTYPQTPLHLQVPPCQVG IFLDYEAGMVSFYNITDHGSLIYSFSECAFTGPLRPFFSPGFNDGGKNTAP LTLCPLNIGSOGSTDY (SEQ ID NO:102, UniProt Accession Number P19474. RO52_HUMAN), or a functional fragment thereof or variant thereof with at least 70, 80, 85, 90, 95, or more sequence identity to SEQ ID NO: 102.

Conjugates can be, for example, small molecule drug to assist in the treatment of a targeted disease or disorder.

### C. Structure of Molecules for Targeted Proteolysis

The three core features, optionally including additional domains, can be assembled in any means suitable to carryout the desired target proteolytic function. Thus envisioned are antibodies of various structures including full antibodies and fusion proteins, provided the core features are represented.

In some embodiments, the molecule takes the form of a multispecific antibody. In some embodiments, the multispecific antibody is a bispecific antibody having two binding moieties that target different epitopes. In some embodiments, the multispecific molecule includes one or more additional binding moieties (e.g., a third binding moiety, a fourth binding moiety, (e.g., a trispecific or a tetraspecific molecule), that target three or more different epitopes.

Multispecific antibody molecules can include more than one antigen-binding site, where different sites are specific for different antigens. Multispecific antibody molecules can additionally or alternatively bind more than one (e.g., two or more) epitopes on the same antigen. For example, when two or more binding moieties are present, one can optionally be a binding domain of the cell-penetrating antibody from which the cell-penetrating feature is derived. In some embodiments, two or more binding moieties target two or more different epitopes of the same target protein. In some embodiments, two or more binding moieties targeting two or more different epitopes of different target proteins. Thus, in some embodiments, the molecule simultaneously targets two or more different proteins for degredation.

In some embodiments, the molecules are heterodimeric bi- and tri-(or more) specific Ig antibodies and Fc fusion proteins. Exemplary structures include, but are not limited to, IgG, IgG, IgM, mono-, di-, tri-, or more scFv-Fcs. For example, bispecific, trispecific, and multispecific formats include, but are not limited to, bispecific and trispecific IgG, IgG-scFv, IgG-dAb, scFv-Fc-scFv, knob-in-hole (KIH)-IgG, KA-body, KIH0Fc-Fab/scFv, tandom scFv, KIH trispecific, bispecific Fc fusion (N- or C-terminal, with or without KIH).

In some embodiments, the multispecific antibody molecule is a bispecific antibody molecule. Bispecific antibody molecules can be classified into different structural groups including having one or more of (i) bispecific immunoglobulin G (BsIgG); (ii) IgG appended with an additional antigen-binding moiety; (iii) bispecific fusion proteins; and (iv) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, kappa-lamda-body, orthogonal Fab. See Spiess et al. Mol. Immunol. 67 (2015): 95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG incudes heavy chains that are engineered for heterodimerization. For example, heavy chains can be engineered for heterodimerization using a “knobs-into-holes” strategy, a SEED platform, a common heavy chain (e.g., in Kk-bodies), and use of heterodimeric Fc regions. See Spiess et al. Mol. Immunol. 67 (2015): 95-106. Strategies that have been used to avoid heavy chain pairing of homodimers in BslgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of multispecific antibody molecules, including bispecific antibody molecules. For example, bivalent monospecific IgG and monovalent bispecific IgG can be engineered to have bi- or more specificity by appending an additional antigen-binding unit onto the IgG, e.g., at the N- or C-terminus of either the heavy or light chain. Exemplary additional antigen-binding units include single domain antibodies (e.g., variable heavy chain or variable light chain), engineered protein scaffolds, and paired antibody variable regions (e.g., single chain variable fragments or variable fragments). See Id. Examples of appended IgG formats include dual variable domain IgG (DVD-Ig), IgG (H)-scFv, scFv-(H) IgG, IgG (L)-scFv, scFv-(L) IgG, IgG (L,H)-Fv, IgG (H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67 (2015): 95-106. An example of an IgG-scFv is MM-141 (Merrimack Pharmaceuticals), which binds IGF-1R and HER3. Examples of DVD-Ig include ABT-981 (AbbVie), which binds IL-1 alpha and IL-1beta; and ABT-122 (AbbVie), which binds TNF and IL-17A.

Bispecific fusion proteins include antibody fragments (nanobody, nanobody-HAS, BITE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, intrabody) linked to other proteins, e.g., and Fc domain, e.g., to add additional functionality.

Chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

In some embodiments the multispecific molecule includes a heavy chain constant region (e.g., an Fc region) chosen from the heavy chain constant regions of IgG1, IgG2, and IgG4, more particularly, the heavy chain constant region of human IgG1, IgG2 or IgG4. In some embodiments, one or two antigen binding moieties (or a VH thereof) is linked to a heavy chain constant region (e.g., one or more of CH1, hinge, CH2, CH3, an Fc region, etc.), e.g., covalently linked. In some embodiments, one or both antigen binding moieties (or a VL thereof) is linked to a light chain constant region (e.g., CL), e.g., covalently linked. In some embodiments, the CH3 and/or CL is further linked, e.g., covalently linked, to an additional antigen binding moiety.

In some embodiments, the heavy chain constant region (e.g., an Fc region) is altered, e.g., mutated, to increase or decrease one or more of: Fc receptor binding, antibody glycosylation, the number of cysteine residues, effector cell function, or complement function.

In some embodiments, an interface of a first and second heavy chain constant regions (e.g., Fc region) is altered, e.g., mutated, to increase or decrease dimerization, e.g., relative to a non-engineered interface. In some embodiments, the dimerization of the heavy chain constant region (e.g., Fc region) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface. In some embodiments, the heavy chain constant region (e.g., Fc region) comprises an amino acid substitution at a position chosen from one or more of 347, 349, 350, 351, 366, 368, 370, 392, 394, 395, 397, 398, 399, 405, 407, or 409, e.g., of the Fc region of human IgG1, numbered based on the Eu numbering system. In some embodiments, the heavy chain constant region (e.g., Fc region) includes an amino acid substitution chosen from: T366S, L368A, or Y407V (e.g., corresponding to a cavity or hole), or T366W (e.g., corresponding to a protuberance or knob), or a combination thereof, numbered based on the Eu numbering system.

In some embodiments, the heavy chain constant region (e.g., an Fc region) includes one or more mutations that increase or decrease one or more of: Fc receptor binding, antibody glycosylation, the number of cysteine residues, effector cell function, or complement function, relative to a naturally-existing heavy chain constant region.

In some embodiments, a multispecific antibody is formed of two or more parts, wherein the first part of the molecule includes a first heavy chain constant region (e.g., a first Fc region) and the second part of the molecule includes a second heavy chain constant region (e.g., a second Fc region), wherein the first heavy chain constant region includes one or more mutations that increase heterodimerization of the first heavy chain constant region and the second heavy chain constant region, relative to a naturally-existing heavy chain constant region, and/or wherein the second heavy chain constant region includes one or more mutations that increase heterodimerization of the second heavy chain constant region and the first heavy chain constant region, relative to a naturally-existing heavy chain constant region. In some embodiments, the first and the second heavy chain constant regions (e.g., first and second Fc regions) include one or more of: a paired cavity-protuberance (“knob-in-a hole”), an electrostatic interaction, or a strand-exchange, such that a greater ratio of heteromultimer:homomultimer forms, e.g., relative to naturally-existing heavy chain constant regions. In some embodiments, the first and/or second heavy chain constant region (e.g., a first and/or second Fc region, e.g., a first and/or second IgG1 Fc region) incudes an amino acid substitution at a position chosen from one or more of 347, 349, 350, 351, 366, 368, 370, 392, 394, 395, 397, 398, 399, 405, 407, or 409, numbered based on the Eu numbering system. In some embodiments, the first and/or second heavy chain constant region (e.g., a first and/or second Fc region, e.g., a first and/or second IgG1 Fc region) comprises an amino acid substitution chosen from: T366S, L368A, Y407V, or Y349C (e.g., corresponding to a cavity or hole), or T366W or S354C (e.g., corresponding to a protuberance or knob), or a combination thereof, numbered based on the Eu numbering system.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. The DNA sequence will then be expressed by a cell as a single protein. The protein can be engineered to include the full sequence of both original proteins, or only a portion of either. If the two entities are proteins, often linker (or “spacer”) peptides are also added which make it more likely that the proteins fold independently and behave as expected.

The function of the antibody may be enhanced by coupling the antibody or a fragment thereof with a therapeutic agent. Such coupling of the antibody or fragment with the therapeutic agent can be achieved by making an immunoconjugate or by making a fusion protein, or by linking the antibody or fragment to a nucleic acid such as DNA or RNA (e.g., siRNA), comprising the antibody or antibody fragment and the therapeutic agent.

In some embodiments, the antibody is modified to alter its half-life. In some embodiments, it is desirable to increase the half-life of the antibody so that it is present in the circulation or at the site of treatment for longer periods of time. For example, it may be desirable to maintain titers of the antibody in the circulation or in the location to be treated for extended periods of time. In other embodiments, the half-life of the antibody is decreased to reduce potential side effects. Antibody fragments, may have a shorter half-life than full size antibodies. Other methods of altering half-life are known and can be used in the described methods. For example, antibodies can be engineered with Fc variants that extend half-life, e.g., using Xtend™ antibody half-life prolongation technology (Xencor, Monrovia, CA).

### III. Exemplary Cell-Penetrating Antibody Sequences

Although generally referred to herein as “3E10” or “3E10 antibodies,” “5C6” or “5C6 antibodies” it will be appreciated that fragments and binding proteins, including antigen-binding fragments, variants, and fusion proteins such as scFv, di-scFv, tri-scFv, and other single chain variable fragments, and other cell-penetrating, nucleic acid transporting molecules disclosed herein are encompassed by the phrase are also expressly provided for use in compositions and methods disclosed herein. Thus, the antibodies and other binding proteins are also referred to herein as cell-penetrating.

### A. 3E10 Antibodies

In preferred embodiments, the 3E10 antibody is transported into the cytoplasm and/or nucleus of the cells without the aid of a carrier or conjugate. For example, the monoclonal antibody 3E10 and active fragments thereof that are transported in vivo to the nucleus of mammalian cells without cytotoxic effect are disclosed in U.S. Pat. Nos. 4,812,397 and 7,189,396 to Richard Weisbart.

In some embodiments, the antibody may bind and/or inhibit Rad51. See for example, the antibody described in Turchick, et al., Nucleic Acids Res., 45 (20): 11782-11799 (2017), WO 2020/047344, and WO 2020/047353, each of which is specifically incorporated by reference herein, in its entirety.

The 3E10 antibody is typically a monoclonal 3E10, or a variant, derivative, fragment, fusion, or humanized form thereof that binds the same or different epitope(s) as 3E10.

A deposit according to the terms of the Budapest Treaty of a hybridoma cell line producing monoclonal antibody 3E10 was received on Sep. 6, 2000, and accepted by, American Type Culture Collection (ATCC), 10801 University Blvd., Manassas, VA 20110-2209, USA, and given Patent Deposit Number PTA-2439.

Thus, the antibody may have the same or different epitope specificity as monoclonal antibody 3E10 produced by ATCC No. PTA 2439 hybridoma. The antibody can have the paratope of monoclonal antibody 3E10. The antibody can be a single chain variable fragment of 3E10, or a variant, e.g., a conservative variant thereof. For example, the antibody can be a single chain variable fragment of 3E10 (3E10 Fv), or a variant thereof.

### 1. 3E10 Sequences

Amino acid sequences of monoclonal antibody 3E10 are known in the art. For example, sequences of the 3E10 heavy and light chains are provided below, where single underlining indicates the CDR regions identified according to the Kabat system, and in SEQ ID NOS: 12-14, 103-106, and 118 italics indicates the variable regions and double underlining indicates the signal peptide. CDRs according to the IMGT system are also provided. All of the sequences are disclosed with and with the signal peptide, and each heavy and light chain variable region is expressly disclosed both with and without a constant region. Thus, the heavy and light chain variable regions (and CDRs thereof) of SEQ ID NOS: 12-14, 103-106, and 118 are also expressly provided free from their constant regions, and can be used to form the binding fragments and fusion proteins provided herein with a substitute constant region or no constant region.

### a. 3E10 Heavy Chain

In some embodiments, a heavy chain variable region of 3E10 is:

(SEQ ID NO: 1; Zack, et al., Immunology and Cell
Biology, 72:513-520 (1994); GenBank: L16981.1 -
Mouse Ig rearranged L-chain gene, partial cds;
and GenBank: AAA65679.1 - immunoglobulin
heavy chain, partial [Mus musculus])
EVQLVESGGGLVKPGGSRKLSCAASGFTFSDYGMHWVRQAPEKGLEWVAYI
SSGSSTIYYADTVKGRFTISRDNAKNTLFLQMTSLRSEDTAMYYCARRGLL
LDYWGQGTTLTVSS.

In some embodiments, a 3E10 heavy chain is expressed as

(3E10 WT Heavy Chain; SEQ ID NO: 12)
MGWSCIILFLVATATGVHSEVQLVESGGGLVKPGGSRKLSCAASGFTFSD
YGMHWVRQAPEKGLEWVAYISSGSSTIYYADTVKGRFTISRDNAKNTLFL
QMTSLRSEDTAMYYCARRGLLLDYWGQGTTLTVSAASTKGPSVFPLAPSS
KSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAP
ELLGGPSVFLFPPKPKDILMISRTPEVTCVVVDVSHEDPEVKENWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK,
or
(3E10 WT, CrossmAb Heavy Chain; SEQ ID NO: 118)
MGWSCIILFLVATATGVHSEVQLVESGGGLVKPGGSRKLSCAASGFTFSD
YGMHWVRQAPEKGLEWVAYISSGSSTIYYADTVKGRFTISRDNAKNTLFL
QMTSLRSEDTAMYYCARRGLLLDYWGQGTTLTVSAASTKGPSVFPLAPSS
KSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLWCLVKGFYPSDIAVEWE
SNGQPENNYKTTPPVLDSDGSFFLYSKLTVDKSRWQQGNVFSCSVMHEAL
HNHYTQKSLSLSPGK.

Variants of the 3E10 antibody which incorporate mutations into the wild type sequence are also known in the art, as disclosed for example, in Zack, et al., J. Immunol., 157 (5): 2082-8 (1996). For example, amino acid position 31 of the heavy chain variable region of 3E10 has been determined to be influential in the ability of the antibody and fragments thereof to penetrate nuclei and bind to DNA (bolded in SEQ ID NOS: 1, 2 and 13). A D31N mutation (bolded in SEQ ID NOS: 2 and 13) in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)). In some embodiments, the antibody has the D31N substitution.

In some embodiments, an amino acid sequence for a preferred variant of a heavy chain variable region of 3E10 is:

(SEQ ID NO: 2)
EVQLVESGGGLVKPGGSRKLSCAASGFTFSNYGMHWVRQAPEKGLEWVAYI
SSGSSTIYYADTVKGRFTISRDNAKNTLFLQMTSLRSEDTAMYYCARRGLL
LDYWGQGTTLTVSS

Other heavy chain variable regions include, but are not limited to:

(SEQ ID NO: 111)
EVQLVESGGGLVKPGGSRKLSCAASGFTFSRYGMHWVRQAPEKGLEWVAYI
SSGSSTIYYADTVKGRFTISRDNAKNTLFLQMTSLRSEDTAMYYCARRGLL
LDYWGQGTTLTVS,
and
(SEQ ID NO: 112)
EVQLVESGGGLVKPGGSRKLSCAASGFTFSKYGMHWVRQAPEKGLEWVAYI
SSGSSTIYYADTVKGRFTISRDNAKNTLFLQMTSLRSEDTAMYYCARRGLL
LDYWGQGTTLTVS

In some embodiments, a 3E10 heavy chain is expressed as

(3E10 D31N Variant Heavy Chain; SEQ ID NO: 13)
MGWSCIILFLVATATGVHSEVQLVESGGGLVKPGGSRKLSCAASGFTFS
NYGMHWVRQAPEKGLEWVAYISSGSSTIYYADTVKGRFTISRDNAKNTL
FLQMTSLRSEDTAMYYCARRGLLLDYWGQGTTLTVSAASTKGPSVFPLA
PSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLIVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGK.

In some embodiments, the C-terminal serine of SEQ ID NOS: 1 or 2 is absent or substituted, with, for example, an alanine, in 3E10 heavy chain variable region.

In some embodiments, a 3E10 heavy chain is expressed as

(SEQ ID NO: 103)
MGWSCIILFLVATATGVHSEVQLVESGGGLVKPGGSRKLSCAASGFTFS
RYGMHWVRQAPEKGLEWVAYISSGSSTIYYADTVKGRFTISRDNAKNTL
FLQMTSLRSEDTAMYYCARRGLLLDYWGQGTTLTVSAASTKGPSVEPLA
PSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLIVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGK,
or
(SEQ ID NO: 104)
MGWSCIILFLVATATGVHSEVQLVESGGGLVKPGGSRKLSCAASGFTFS
KYGMHWVRQAPEKGLEWVAYISSGSSTIYYADTVKGRFTISRDNAKNTL
FLQMTSLRSEDTAMYYCARRGLLLDYWGQGTTLTVSAASTKGPSVFPLA
PSSKSTSGGTAALGCLVKDYFPEPVIVSWNSGALTSGVHTFPAVLQSSG
LYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGK

The complementarity determining regions (CDRs) as identified by Kabat are shown with underlining above and include CDR H1.1 (original sequence): DYGMH (SEQ ID NO:15); CDR H1.2 (with D31N mutation): NYGMH (SEQ ID NO:16); CDR H2.1: YISSGSSTIYYADTVKG (SEQ ID NO: 17); CDR H3.1: RGLLLDY (SEQ ID NO:18).

Other variant of Kabat CHR H1.1 and H1.2 include CDR H1.3 (with D31R mutation): RYGMH (SEQ ID NO:107); and CDR H1.4 (with D31K mutation) KYGMH (SEQ ID NO:108).

Variants of Kabat CDR H2.1 include YISSGSSTIYYADSVKG (SEQ ID NO: 19) and YISSSSSTIYYADSVKG (SEQ ID NO:42).

Additionally, or alternatively, the heavy chain complementarity determining regions (CDRs) can be defined according to the IMGT system. The complementarity determining regions (CDRs) as identified by the IMGT system include CDR H1.3 (original sequence): GFTFSDYG (SEQ ID NO: 20); CDR H1.4 (with D31N mutation): GFTFSNYG (SEQ ID NO:21); GFTFSRYG (SEQ ID NO:109); CDR H1.4 (with D31K mutation) GFTFSKYG (SEQ ID NO:110); CDR H2.2: ISSGSSTI (SEQ ID NO:22) and variant ISSSSSTI (SEQ ID NO:43); CDR H3.2: ARRGLLLDY (SEQ ID NO: 23).

### b. 3E10 Light Chain

In some embodiments, a light chain variable region of 3E10 is:

(SEQ ID NO: 7)
DIVLTQSPASLAVSLGQRATISCRASKSVSTSSYSYMHWYQQKPGQPPKLL
IKYASYLESGVPARFSGSGSGTDFTLNIHPVEEEDAATYYCQHSREFPWTF
GGGTKLEIK

An amino acid sequence for the light chain variable region of 3E10 can also be:

(SEQ ID NO: 8)
DIVLTQSPASLAVSLGQRATISCRASKSVSTSSYSYMHWYQQKPGQPPKLL
IKYASYLESGVPARFSGSGSGTDFHLNIHPVEEEDAATYYCQHSREFPWTF
GGGTKLELK
or
(SEQ ID NO: 116)
DIVLTQSPASLAVSLGQRATISCRASKSVDTSSYSYMHWYQQKPGQPPKLL
IKYASYLESGVPARFSGSGSGTDFTLNIHPVEEEDAATYYCQHSREFPWTF
GGGTKLEIKR,
or
(SEQ ID NO: 117)
DIVLTQSPASLAVSLGQRATISCRASKSVDTSSYSYMHWYQQKPGQPPKLL
IKYASYLESGVPARFSGSGSGTDFTLNIHPVEEEDAATYYCQHSREFPWTF
GGGTKLEIK,
or
(SEQ ID NO: 128)
DIVLTQSPASLAVSLGQRATISCRASKSVSTSSYSYMHWYQQKPGQPPKLL
IKYASYLESGVPARFSGSGSGIDFTLNIHPVEEEDAATYYCQHSNEFPWTF
GGGTKLEIKR.

In some embodiments, a 3E10 light chain is expressed as

(3E10 WT Light Chain; SEQ ID NO: 14)
MGWSCIILFLVATATGVHSDIVLTQSPASLAVSLGQRATISCRASKSVSTS
SYSYMHWYQQKPGQPPKLLIKYASYLESGVPARFSGSGSGTDFTLNIHPVE
EEDAATYYCQHSREFPWTFGGGTKLEIKRTVAAPSVFIFPPSDEQLKSGTA
SVVCLLNNFYPREAKVQWKVDNALQSGNSQESVTEQDSKDSTYSLSSTLTL
SKADYEKHKVYACEVTHQGLSSPVTKSFNRGEC,
or
(SEQ ID NO: 105)
MGWSCIILFLVATATGVHSDIVLTQSPASLAVSLGQRATISCRASKSVDTS
SYSYMHWYQQKPGQPPKLLIKYASYLESGVPARFSGSGSGTDFTLNIHPVE
EEDAATYYCQHSREFPWTFGGGTKLEIKRTVAAPSVFIFPPSDEQLKSGTA
SVVCLLNNFYPREAKVQWKVDNALQSGNSQESVTEQDSKDSTYSLSSTLTL
SKADYEKHKVYACEVTHQGLSSPVTKSFNRGEC,
or
(SEQ ID NO: 106)
MGWSCIILFLVATATGVHSDIVLTQSPASLAVSLGQRATISCRASKSVSTS
SYSYMHWYQQKPGQPPKLLIKYASYLESGVPARFSGSGSGTDFTLNIHPVE
EEDAATYYCQHSNEFPWTFGGGTKLEIKRTVAAPSVFIFPPSDEQLKSGTA
SVVCLLNNFYPREAKVQWKVDNALQSGNSQESVTEQDSKDSTYSLSSTLTL
SKADYEKHKVYACEVTHQGLSSPVTKSFNRGEC.

Other 3E10 light chain sequences are known in the art. See, for example, Zack, et al., J. Immunol., 15; 154 (4): 1987-94 (1995); GenBank: L16981.1—Mouse Ig rearranged L-chain gene, partial cds; GenBank: AAA65681.1—immunoglobulin light chain, partial [Mus musculus]).

The complementarity determining regions (CDRs) as identified by Kabat are shown with underlining, including CDR L1.1: RASKSVSTSSYSYMH (SEQ ID NO:24); CDR L2.1: YASYLES (SEQ ID NO: 25); CDR L3.1: QHSREFPWT (SEQ ID NO:26).

Variants of Kabat CDR L1.1 include RASKSVSTSSYSYLA (SEQ ID NO: 27), RASKTVSTSSYSYMH (SEQ ID NO:44), and CDR L1.2: RASKSVDTSSYSYMH (SEQ ID NO:113).

A variant of Kabat CDR L2.1 is YASYLQS (SEQ ID NO:28).

A variant of Kabat CDR L3.1 is CDR L3.3: QHSNEFPWT (SEQ ID NO: 114).

Additionally, or alternatively, the heavy chain complementarity determining regions (CDRs) can be defined according to the IMGT system. The complementarity determining regions (CDRs) as identified by the IMGT system include CDR L1.2 KSVSTSSYSY (SEQ ID NO:29) and variants CDR L1.2 KTVSTSSYSY (SEQ ID NO:45) and KSVDTSSYSY (SEQ ID NO: 115); CDR L2.2: YAS (SEQ ID NO:30); CDR L3.2: QHSREFPWT (SEQ ID NO: 26); CDR L3.3: QHSNEFPWT (SEQ ID NO:114).

In some embodiments, the C-terminal end of sequence of SEQ ID NOS: 7 or 8 further includes an arginine in the 3E10 light chain variable region.

### 2. Humanized 3E10

In some embodiments, the antibody is a humanized antibody. Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

Exemplary 3E10 humanized sequences are discussed in WO 2015/106290, WO 2016/033324, WO 2019/018426, and WO/2019/018428, and provided below.

a. Humanized 3E10 Heavy Chain Variable Regions

In some embodiments, a humanized 3E10 heavy chain variable domain includes

(hVH1, SEQ ID NO: 3)
EVQLVQSGGGLIQPGGSLRLSCAASGFTFSNYGMHWVRQAPGKGLEWVSYI
SSGSSTIYYADSVKGRFTISRDNSKNTLYLQMNSLRAEDTAVYYCARRGLL
LDYWGQGTTVTVSS,
or
(hVH2, SEQ ID NO: 4)
EVQLVESGGGLIQPGGSLRLSCAASGFTFSNYGMHWVRQAPGKGLEWVSYI
SSGSSTIYYADSVKGRFTISRDNSKNTLYLQMTSLRAEDTAVYYCARRGLL
LDYWGQGTTLTVSS,
or
(hVH3, SEQ ID NO: 5)
EVQLQESGGGVVQPGGSLRLSCAASGFTFSNYGMHWIRQAPGKGLEWVSYI
SSGSSTIYYADSVKGRFTISRDNSKNTLYLQMNSLRSEDTAVYYCARRGLL
LDYWGQGTLVTVSS,
or
(hVH4, SEQ ID NO: 6)
EVQLVESGGGLVQPGGSLRLSCSASGFTFSNYGMHWVRQAPGKGLEYVSYI
SSGSSTIYYADTVKGRFTISRDNSKNTLYLQMSSLRAEDTAVYYCVKRGLL
LDYWGQGTLVTVSS,
or
(variants 2, 6 and 10, SEQ ID NO: 46)
EVQLVESGGGLVQPGGSLRLSCAASGFTFSNYGMHWVRQAPGKGLEWVSYI
SSSSSTIYYADSVKGRFTISRDNAKNSLYLQMNSLRAEDTAVYYCARRGLL
LDYWGQGTTVTVSS,
or
(variants 3, 7 and 11, SEQ ID NO: 47)
EVQLVESGGGVVQPGGSLRLSCAASGFTFSNYGMHWVRQAPEKGLEWVSYI
SSSSSTIYYADSVKGRFTISRDNSKNTLYLQMNSLRAEDTAVYYCARRGLL
LDYWGQGTTVTVSS,
or
(variants 4, 8 and 12, SEQ ID NO: 48)
EVQLVESGGGDVKPGGSLRLSCAASGFTFSNYGMHWVRQAPEKGLEWVSYI
SSSSSTIYYADSVKGRFTISRDNSKNTLYLQMNSLRAEDTAVYYCARRGLL
LDYWGQGTTVTVSS,
or
(variants 13, 16 and 19, SEQ ID NO: 50)
EVQLVESGGGLVQPGGSLRLSCAASGFTFSNYGMHWVRQAPGKGLEWVSYI
SSGSSTIYYADSVKGRFTISRDNAKNSLYLQMNSLRAEDTAVYYCARRGLL
LDYWGQGTTVTVSS,
or
(variants 14 and 17, SEQ ID NO: 51)
EVQLVESGGGVVQPGGSLRLSCAASGFTFSNYGMHWVRQAPEKGLEWVSYI
SSGSSTIYYADSVKGRFTISRDNSKNTLYLQMNSLRAEDTAVYYCARRGLL
LDYWGQGTTVTVSS,
or
(variants 15 and 18, SEQ ID NO: 52)
EVQLVESGGGDVKPGGSLRLSCAASGFTFSNYGMHWVRQAPEKGLEWVSYI
SSGSSTIYYADSVKGRFTISRDNSKNTLYLQMNSLRAEDTAVYYCARRGLL
LDYWGQGTTVTVSS.

b. Humanized 3E10 Light Chain Variable Regions

In some embodiments, a humanized 3E10 light chain variable domain includes

(hVL1, SEQ ID NO: 9)
DIQMTQSPSSLSASVGDRVTITCRASKSVSTSSYSYLAWYQQKPEKAPKLL
IKYASYLQSGVPSRFSGSGSGTDFTLTISSLQPEDFATYYCQHSREFPWTF
GAGTKLELK,
or
(hVL2, SEQ ID NO: 10)
DIQMTQSPSSLSASVGDRVTISCRASKSVSTSSYSYMHWYQQKPEKAPKLL
IKYASYLQSGVPSRFSGSGSGTDFTLTISSLQPEDVATYYCQHSREFPWTF
GAGTKLELK,
or
(hVL3, SEQ ID NO: 11)
DIVLTQSPASLAVSPGQRATITCRASKSVSTSSYSYMHWYQQKPGQPPKLL
IYYASYLESGVPARFSGSGSGTDFTLTINPVEANDTANYYCQHSREFPWTF
GQGTKVEIK
(variants 2, 3 and 4, SEQ ID NO: 53)
DIQMTQSPSSLSASLGDRATITCRASKSVSTSSYSYMHWYQQKPGQPPKLL
IKYASYLESGVPSRFSGSGSGTDFTLTISSLQPEDAATYYCQHSREFPWTF
GGGTKVEIK
(variants 6, 7 and 8, SEQ ID NO: 54)
DIQMTQSPSSLSASLGDRATITCRASKSVSTSSYSYMHWYQQKPGQAPKLL
IKYASYLESGVPSRFSGSGSGTDFTLTISSLQPEDFATYYCQHSREFPWTF
GQGTKVEIK
(variants 10, 11 and 12, SEQ ID NO: 55)
DIQMTQSPSSLSASVGDRVTITCRASKSVSTSSYSYMHWYQQKPGKAPKLL
IKYASYLESGVPSRFSGSGSGTDFTLTISSLQPEDFATYYCQHSREFPWTF
GQGTKVEIK
(variants 13, 14 and 15, SEQ ID NO: 56)
DIQMTQSPSSLSASLGDRATITCRASKTVSTSSYSYMHWYQQKPGQPPKLL
IKYASYLESGVPSRFSGSGSGTDFTLTISSLQPEDAATYYCQHSREFPWTF
GGGTKVEIK
(variants 16, 17 and 18, SEQ ID NO: 57)
DIQMTQSPSSLSASVGDRVTITCRASKTVSTSSYSYMHWYQQKPGKAPKLL
IKYASYLESGVPSRFSGSGSGTDFTLTISSLQPEDFATYYCQHSREFPWTF
GQGTKVEIK
(variant 19, SEQ ID NO: 58)
DIQMTQSPSSLSASLGDRATITCRASKTVSTSSYSYMHWYQQKPGQAPKLL
IKYASYLESGVPSRFSGSGSGTDFTLTISSLQPEDFATYYCQHSREFPWTF
GQGTKVEIK

c. Cell Penetration and Nuclear Localization

The disclosed compositions and methods typically utilize antibodies that maintain the ability to penetrate cells, and optionally nuclei.

Mutations in 3E10 that interfere with its ability to bind DNA may render the antibody incapable of nuclear penetration. Thus, typically the disclosed variants and humanized forms of the antibody maintain the ability to bind nucleic acids, particularly DNA. In addition, 3E10 scFv has previously been shown capable of penetrating into living cells and nucleic in an ENT2-dependent manner, with efficiency of uptake impaired in ENT2-deficient cells (Hansen, et al., J. Biol. Chem. 282, 20790-20793 (2007)). Thus, in some embodiments, the disclosed variants and humanized forms of the antibody maintain the ability penetrate into cell nuclei in an ENT-dependent, preferably ENT2-dependent manner.

As discussed in WO 2019/152806 and WO 2019/152808 some humanized 3E10 variant were found to penetrate cell nuclei more efficiently than the original murine 3E10 (D31N) di-scFv, while others were found to have lost the ability to penetrate nuclei. In particular, variants 10 and 13 penetrated nuclei very well compared to the murine antibody.

Potential bipartite nuclear localization signals (NLS) in humanized 3E10 VL have been identified and may include part or all of the following sequences:

(SEQ ID NO: 88)
RASKSVSTSSYSYMHWYQQKPGQPPKLLIKY;
(SEQ ID NO: 89)
RASKTVSTSSYSYMHWYQQKPGQPPKLLIKY;
or
(SEQ ID NO: 90)
RVTITCRASKSVSTSSYSYMHWYQQKPGKAPKL.

An exemplary consensus NLS can be, or include, (X)RASKTVSTSSYSYMHWYQQKPGOPPKLL(X)KY (where (X)=any residue, but preferentially is a basic residue (R or K) (SEQ ID NO:91) or a variant thereof with at least 60, 65, 70, 75, 80, 85, 90, 95, 96, 97, 98, 99 percent sequence identity to SEQ ID NO:53.

Thus, in some embodiments, particularly where nuclear importation is important, the disclosed antibodies may include the sequence of any one of SEQ ID NOS: 88-91, or fragments and variants thereof (e.g., 70, 75, 80, 85, 90, 95, 96, 97, 98, 99, or 100% amino acid sequence identity with any one of SEQ ID NOS: 88-91) that can translocate into the nucleus of a cell.

Presence of an NLS indicates that 3E10 may cross the nuclear envelope via the nuclear import pathway. In some embodiments, the NLS improves importation by interacting with one or more members of the import pathway. Thus, in some embodiments, the NLS can bind to importin-β, an importin-β/importin-α heterodimer, or a combination thereof.

### 3. Nucleic Acid Binding

The disclosed compositions and methods optionally utilize antibodies that maintain the ability to bind nucleic acids such as DNA, RNA, or a combination thereof.

Molecular modeling of 3E10 (Pymol) revealed a putative Nucleic Acid Binding pocket (NAB1), and illustrated with underlining the sequences below.

WT HEAVY CHAIN scFv SEQUENCE
(SEQ ID NO: 92)
E VQLVESGGGL VKPGGSRKLS CAASGFTFSD YGMHWVRQAP
EKGLEWVAYI SSGSSTIYYA DTVKGRFTIS RDNAKNTLFL
QMTSLRSEDT AMYYCARRGL LLDYWGQGTT LTVS
LIGHT CHAIN scFv SEQUENCE
(SEQ ID NO: 93)
D IVLTQSPASL AVSLGQRATI SCRASKSVST SSYSYMHWYQ
QKPGQPPKLL IKYASYLESG VPARFSGSGS GTDFTLNIHP
VEEEDAATYY CQHSREFPWT FGGGTKLEIK RADAAPGGGG
SGGGGSGGGGS

In some embodiments, the disclosed antibodies include some or all of the underlined NAB1 sequences. In some embodiments, the antibodies include a variant sequence that has an altered ability of bind nucleic acids. In some embodiments, the mutations (e.g., substitutions, insertions, and/or deletions) in the NAB1 improve binding of the antibody to nucleic acids such as DNA, RNA, or a combination thereof. In some embodiments, the mutations are conservative substitutions. In some embodiments, the mutations increase the cationic charge of the NAB1 pocket.

As discussed and exemplified herein, mutation of aspartic acid at residue 31 of CDR1 to asparagine increased the cationic charge of this residue and enhanced nucleic acid binding and delivery in vivo (3E10-D31N).

Additional exemplary variants include mutation of aspartic acid at residue 31 of CDR1 to arginine (3E10-D31R), which modeling indicates expands cationic charge, or lysine (3E10-D31K) which modeling indicates changes charge orientation. Thus, in some embodiments, the 3E10 binding protein includes a D31R or D31K substitution.

Additional exemplary variants include mutation of arginine (R) 96 to asparagine (N), and/or serine(S) 30 to aspartic acid (D) alone or in combination with D31N, D31R, or D31K. Variants having the mutation of arginine (R) 96 to asparagine (N) may have reduced cell penetration, thus in some embodiments, the antibody does not have an R96N mutation.

All of the sequences disclosed herein having the residue corresponding to 3E10 D31 or N31, are expressly disclosed with a D31R or D31K or N31R or N31K substitution.

Molecular modeling of 3E10 (Pymol) revealed a putative Nucleic Acid Binding pocket (NAB1). Mutation of aspartic acid at residue 31 of CDR1 to asparagine increased the cationic charge of this residue and enhanced nucleic acid binding and delivery in vivo (3E10-D31N).

Mutation of aspartic acid at residue 31 of CDR1 to arginine (3E10-D31R), further expanded the cationic charge while mutation to lysine (3E10-D31K) changed charge orientation.

NAB1 amino acids predicted from molecular modeling have been underlined in the heavy and light chain sequences above.

All of the sequences disclosed herein having the residue corresponding with R96 are expressly disclosed with R96N substitution.

All of the sequence disclosed herein having the residue corresponding to S30 are expressly disclosed with S30D.

Any of the substitutions can be included in any combination. Thus sequences having two or three substitution at any combination of residues 31, 30, and 96 are expressly provided.

In particular embodiments, the sequence has 31N, 31K, or 31R alone or in combination with 30D, and without the R96N substitution. Thus, in some embodiments, the residue corresponding to 96 is not N, and in more specific embodiments remains R.

### B. 5C6 Antibody Sequences

mAb 5C6 is a lupus autoantibody that selectively suppressed growth of BRCA2⁽⁻⁾ DLD1 cells. In certain embodiments, recombinant antibodies have the same or similar epitope specificity as IgG2a-k mAb 5C6 produced by MRL/MpJ-Fas^{lpr} mice. This can be achieved by producing a recombinant antibody that contains the paratope of monoclonal antibody 5C6.

### 1. 5C6 Light Chain Variable Region

An amino acid sequence for the kappa light chain variable region (VL) of mAb 5C6 is:

(SEQ ID NO: 94)
DIVLTQSPASLAAVSLGERATISYRASKSVSTSGYSYMHWNQQKPGQA
PRLLIYLVSNLESGVPARFSGSGSGTDFTLNIHPVEEEDAATYYCQHI
RELDTFFGGGTKLEIK.

The complementarity determining regions (CDRs) are shown with underlining, including CDR L1: RASKSVSTSGYSYMH (SEQ ID NO:95); CDR L2: LVSNLES (SEQ ID NO:96); CDR L3: QHIRELDTF (SEQ ID NO: 97).

### 2. 5C6 Heavy Chain Variable Region

An amino acid sequence for the heavy chain variable region (VH) of mAb 5C6 is:

(SEQ ID NO: 98)
QLKLVESGGGLVKPGGSLKLSCAASGFTFSSYTMSWVRQTPAKRLEWV
ATISSGGGSTYYPDSVKGRFTISRDNARNTLYLQMSSLRSEDTAMYYC
ARRAYSKRGAMDYWGQGTSVTVSS.

The complementarity determining regions (CDRs) are shown with underlining, including CDR H1: SYTMS (SEQ ID NO:99); CDR H2: TISSGGGSTYYPDSVKG (SEQ ID NO:100); CDR H3: RAYSKRGAMDY (SEQ ID NO:101).

### C. Fragments, Variants, and Fusion Proteins

The anti-DNA antibody can be composed of an antibody fragment or fusion protein including an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, at least 99%, or 100% identical to the amino acid sequence of the variable heavy chain and/or light chain of 3E10, 5C6, or a humanized form thereof.

The cell-penetrating antibody can be composed of an antibody fragment or fusion protein that includes one or more CDR(s) that is at least 45%, at least 50%, at least 55%, at least 60%, at least 65%, at least 70%, at least 75%, at least 80%, at least 85%, at least 90%, at least 95%, at least 99%, or 100% identical to the amino acid sequence of the CDR(s) of 3E10, 5C6, or a variant or humanized form thereof. The determination of percent identity of two amino acid sequences can be determined by BLAST protein comparison. In some embodiments, the antibody includes one, two, three, four, five, or all six of the CDRs of the above-described preferred variable domains.

Preferably, the antibody includes one of each of a heavy chain CDR1, CDR2, and CDR3 in combination with one of each of a light chain CDR1, CDR2, and CDR3.

Predicted complementarity determining regions (CDRs) of the light chain variable sequence for 3E10 and 5C6 are provided above. See also GenBank: AAA65681.1—immunoglobulin light chain, partial [Mus musculus] and GenBank: L34051.1—Mouse Ig rearranged kappa-chain mRNA V-region. Predicted complementarity determining regions (CDRs) of the heavy chain variable sequence for 3E10 are provide above. See also, for example, Zack, et al., Immunology and Cell Biology, 72:513-520 (1994), GenBank Accession number AAA65679.1. Zach, et al., J. Immunol. 154 (4), 1987-1994 (1995) and GenBank: L16982.1—Mouse Ig rearranged H-chain gene, partial cds.

All of the sequences disclosed herein having the residue corresponding to 3E10 D31 or N31, are expressly disclosed with a D31R or D31K or N31R or N31K substitution therein. Thus, in some embodiments, the 3E10 binding protein is a variant of any of the foregoing or following sequences wherein the amino acid residue corresponding to residue 31 of the 3E10 heavy chain is substituted with arginine (R) or lysine (K).

Also included are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific to an antigenic protein of the present disclosure. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

The cell-penetrating antibodies can be modified to improve their therapeutic potential. For example, in some embodiments, the cell-penetrating antibody is conjugated to another antibody specific for a second therapeutic target in the cytoplasm and/or nucleus of a target cell. For example, the cell-penetrating antibody can be a fusion protein containing 3E10 or 5C6 Fv and a single chain variable fragment of a monoclonal antibody that specifically binds the second therapeutic target. In other embodiments, the cell-penetrating antibody is a bispecific antibody having a first heavy chain and a first light chain from 3E10 or 5C6 and a second heavy chain and a second light chain from a monoclonal antibody that specifically binds the second target.

Bispecific antibodies and other binding proteins having a first heavy chain and a first light chain from 3E10 and a second heavy chain and a second light chain from a monoclonal antibody that specifically binds a second target are discussed in Weisbart, et al., Mol. Cancer Ther., 11 (10): 2169-73 (2012), and Weisbart, et al., Int. J. Oncology, 25:1113-8 (2004), and U.S. Patent Application No. 2013/0266570, which are specifically incorporated by reference in their entireties.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies. In some embodiments, the cell-penetrating antibody may contain two or more linked single chain variable fragments of 3E10 or 5C6 (e.g., di-scFv, 3E10 tri-scFv), or conservative variants thereof. In some embodiments, the antibody is a diabody or triabody. Sequences for single and two or more linked single chain variable fragments of 3E10 are provided in WO 2017/218825 and WO 2016/033321.

### 1. Linkers

The term “linker” as used herein includes, without limitation, peptide linkers. The peptide linker can be any size provided it does not interfere with the binding of the epitope by the variable regions. In some embodiments, the linker includes one or more glycine and/or serine amino acid residues. Monovalent single-chain antibody variable fragments (scFvs) in which the C-terminus of one variable domain are typically tethered to the N-terminus of the other variable domain via a 15 to 25 amino acid peptide or linker. The linker is chosen to permit the heavy chain and light chain to bind together in their proper conformational orientation. Linkers in diabodies, triabodies, etc., typically include a shorter linker than that of a monovalent scFv as discussed above. Di-, tri-, and other multivalent scFvs typically include three or more linkers. The linkers can be the same, or different, in length and/or amino acid composition. Therefore, the number of linkers, composition of the linker(s), and length of the linker(s) can be determined based on the desired valency of the scFv as is known in the art. The linker(s) can allow for or drive formation of a di-, tri-, and other multivalent scFv.

For example, a linker can include 4-8 amino acids. In a particular embodiment, a linker includes the amino acid sequence GQSSRSS (SEQ ID NO: 31). In another embodiment, a linker includes 15-20 amino acids, for example, 18 amino acids. In a particular embodiment, the linker includes the amino acid sequence GQSSRSSSGGGSSGGGGS (SEQ ID NO:32). Other flexible linkers include, but are not limited to, the amino acid sequences Gly-Ser, Gly-Ser-Gly-Ser (SEQ ID NO:33), Ala-Ser, Gly-Gly-Gly-Ser (SEQ ID NO: 34), (Gly4-Ser) 2 (SEQ ID NO:35) and (Gly4-Ser) 4 (SEQ ID NO:36), and (Gly-Gly-Gly-Gly-Ser) 3 (SEQ ID NO:37).

Other exemplary linkers include, for example, RADAAPGGGGSGGGGSGGGGS (SEQ ID NO:59) and ASTKGPSVFP LAP LESSGS (SEQ ID NO:60).

2. Exemplary scFv Sequences

Exemplary murine 3E10 scFv sequences, including mono-, di-, and tri-scFv are disclosed in WO 2016/033321, WO 2017/218825, WO 2019/018426, and WO/2019/018428, and provided below. Cell-penetrating antibodies for use in the disclosed compositions and methods include exemplary scFv, and fragments and variants thereof.

The amino acid sequence for scFv 3E10 (D31N) is:

(SEQ ID NO: 38)
AGIHDIVLTQSPASLAVSLGQRATISCRASKSVSTSSYSYMH
WYQQKPGQPPKLLIKYASYLESGVPARFSGSGSGTDFTLNIH
PVEEEDAATYYCQHSREFPWTFGGGTKLEIKRADAAPGGGGS
GGGGSGGGGSEVQLVESGGGLVKPGGSRKLSCAASGFTFSNY
GMHWVRQAPEKGLEWVAYISSGSSTIYYADTVKGRFTISRDN
AKNTLFLQMTSLRSEDTAMYYCARRGLLLDYWGQGTTLTVSS
LEQKLISEEDLNSAVDHHHHHH.

Annotation of scFv Protein Domains with Reference to SEQ ID NO:38
AGIH sequence increases solubility (amino acids 1~4 of SEQ ID NO: 38)·
Vk variable region (amino acids 5-115 of SEQ ID NO:38)·
Initial (6 aa) of light chain CH1 (amino acids 116-121 of SEQ ID NO: 38)·
(GGGGS) 3 (SEQ ID NO:37) linker (amino acids 122-136 of SEQ ID NO: 38)·
VH variable region (amino acids 137-252 of SEQ ID NO:38)·
Myc tag (amino acids 253-268 SEQ ID NO:38)·
His 6 tag (amino acids 269-274 of SEQ ID NO:38)

### Amino Acid Sequence of 3E10 Di-scFv (D31N)

Di-scFv 3E10 (D31N) is a di-single chain variable fragment including 2× the heavy chain and light chain variable regions of 3E10 and wherein the aspartic acid at position 31 of the heavy chain is mutated to an asparagine. The amino acid sequence for di-scFv 3E10 (D31N) is:

(SEQ ID NO: 39)
AGIHDIVLTQSPASLAVSLGQRATISCRASKSVSTSSYSYMHWYQQKPGQP
PKLLIKYASYLESGVPARFSGSGSGTDFTLNIHPVEEEDAATYYCQHSREF
PWTFGGGTKLEIKRADAAPGGGGSGGGGSGGGGSEVQLVESGGGLVKPGGS
RKLSCAASGFTFSNYGMHWVRQAPEKGLEWVAYISSGSSTIYYADTVKGRF
TISRDNAKNTLFLQMTSLRSEDTAMYYCARRGLLLDYWGQGTTLTVSSAST
KGPSVFPLAPLESSGSDIVLTQSPASLAVSLGQRATISCRASKSVSTSSYS
YMHWYQQKPGQPPKLLIKYASYLESGVPARFSGSGSGTDFTLNIHPVEEED
AATYYCQHSREFPWTFGGGTKLEIKRADAAPGGGGSGGGGSGGGGSEVQLV
ESGGGLVKPGGSRKLSCAASGFTFSNYGMHWVRQAPEKGLEWVAYISSGSS
TIYYADTVKGRFTISRDNAKNTLFLQMTSLRSEDTAMYYCARRGLLLDYWG
QGTTLTVSSLEQKLISEEDLNSAVDHHHHHH.

Annotation of di-scFv Protein Domains with Reference to SEQ ID NO:39
AGIH sequence increases solubility (amino acids 1~4 of SEQ ID NO: 39)
Vk variable region (amino acids 5-115 of SEQ ID NO:39)·
Initial (6 aa) of light chain CH1 (amino acids 116-121 of SEQ ID NO: 39)
(GGGGS) 3 (SEQ ID NO:37) linker (amino acids 122-136 of SEQ ID NO: 39)
VH variable region (amino acids 137-252 of SEQ ID NO:39)
Linker between Fv fragments consisting of human IgG CH1 initial 13 amino acids (amino acids 253-265 of SEQ ID NO:39)
Swivel sequence (amino acids 266-271 of SEQ ID NO:39)
Vk variable region (amino acids 272-382 of SEQ ID NO:39)
Initial (6 aa) of light chain CH1 (amino acids 383-388 of SEQ ID NO: 39)
(GGGGS) 3 (SEQ ID NO:37) linker (amino acids 389-403 of SEQ ID NO: 39)
VH variable region (amino acids 404-519 of SEQ ID NO:39)
Myc tag (amino acids 520-535 of SEQ ID NO:39)
His 6 tag (amino acids 536-541 of SEQ ID NO:39)

### Amino Acid Sequence for Tri-scFv

Tri-scFv 3E10 (D31N) is a tri-single chain variable fragment including 3× the heavy chain and light chain variable regions of 310E and wherein the aspartic acid at position 31 of the heavy chain is mutated to an asparagine. The amino acid sequence for tri-scFv 3E10 (D31N) is:

(SEQ ID NO: 40)
AGIHDIVLTQSPASLAVSLGQRATISCRASKSVSTSSYSYMHWYQQKPGQP
PKLLIKYASYLESGVPARFSGSGSGTDFTLNIHPVEEEDAATYYCQHSREF
PWTFGGGTKLEIKRADAAPGGGGSGGGGSGGGGSEVQLVESGGGLVKPGGS
RKLSCAASGFTFSNYGMHWVRQAPEKGLEWVAYISSGSSTIYYADTVKGRF
TISRDNAKNTLFLQMTSLRSEDTAMYYCARRGLLLDYWGQGTTLTVSSAST
KGPSVFPLAPLESSGSDIVLTQSPASLAVSLGQRATISCRASKSVSTSSYS
YMHWYQQKPGQPPKLLIKYASYLESGVPARFSGSGSGTDFTLNIHPVEEED
AATYYCQHSREFPWTFGGGTKLEIKRADAAPGGGGSGGGGSGGGGSEVQLV
ESGGGLVKPGGSRKLSCAASGFTFSNYGMHWVRQAPEKGLEWVAYISSGSS
TIYYADTVKGRFTISRDNAKNTLFLQMTSLRSEDTAMYYCARRGLLLDYWG
QGTTLTVSSASTKGPSVFPLAPLESSGSDIVLTQSPASLAVSLGQRATISC
RASKSVSTSSYSYMHWYQQKPGQPPKLLIKYASYLESGVPARFSGSGSGTD
FTLNIHPVEEEDAATYYCQHSREFPWTFGGGTKLEIKRADAAPGGGGSGGG
GSGGGGSEVQLVESGGGLVKPGGSRKLSCAASGFTFSNYGMHWVRQAPEKG
LEWVAYISSGSSTIYYADTVKGRFTISRDNAKNTLFLQMTSLRSEDTAMYY
CARRGLLLDYWGQGTTLTVSSLEQKLISEEDLNSAVDHHHHHH.

Annotation of Tri-scFv Protein Domains with Reference to SEQ ID NO:40
AGIH sequence increases solubility (amino acids 1~4 of SEQ ID NO: 40)
Vk variable region (amino acids 5-115 of SEQ ID NO:40)
Initial (6 aa) of light chain CH1 (amino acids 116-121 of SEQ ID NO: 40)
(GGGGS) 3 (SEQ ID NO:37) linker (amino acids 122-136 of SEQ ID NO: 40)
VH variable region (amino acids 137-252 of SEQ ID NO:40)
Linker between Fv fragments consisting of human IgG CH1 initial 13 amino acids (amino acids 253-265 of SEQ ID NO:40)
Swivel sequence (amino acids 266-271 of SEQ ID NO:40)
Vk variable region (amino acids 272-382 of SEQ ID NO:40)
Initial (6 aa) of light chain CH1 (amino acids 383-388 of SEQ ID NO: 40)
(GGGGS)₃ (SEQ ID NO:37) linker (amino acids 389-403 of SEQ ID NO: 40)
VH variable region (amino acids 404-519 of SEQ ID NO:40)
Linker between Fv fragments consisting of human IgG C_{H}1 initial 13 amino acids (amino acids 520-532 of SEQ ID NO:40)
Swivel sequence (amino acids 533-538 of SEQ ID NO:40)
Vk variable region (amino acids 539-649 of SEQ ID NO:40)
Initial (6 aa) of light chain CH1 (amino acids 650-655 of SEQ ID NO: 40)
(GGGGS) 3 (SEQ ID NO:37) linker (amino acids 656-670 of SEQ ID NO: 40)
VH variable region (amino acids 671-786 of SEQ ID NO:40)
Myc tag (amino acids 787-802 of SEQ ID NO:40)
His 6 tag (amino acids 803-808 of SEQ ID NO:40)

WO 2016/033321 and Noble, et al., Cancer Research, 75 (11): 2285-2291 (2015), show that di-scFv and tri-scFv have some improved and additional activities compared to their monovalent counterpart. The subsequences corresponding to the different domains of each of the exemplary fusion proteins are also provided above. One of skill in the art will appreciate that the exemplary fusion proteins, or domains thereof, can be utilized to construct fusion proteins discussed in more detail above. For example, in some embodiments, the di-scFv includes a first scFv including a Vk variable region (e.g., amino acids 5-115 of SEQ ID NO:39, or a functional variant or fragment thereof), linked to a VH variable domain (e.g., amino acids 137-252 of SEQ ID NO:39, or a functional variant or fragment thereof), linked to a second scFv including a Vk variable region (e.g., amino acids 272-382 of SEQ ID NO:39, or a functional variant or fragment thereof), linked to a VH variable domain (e.g., amino acids 404-519 of SEQ ID NO: 39, or a functional variant or fragment thereof). In some embodiments, a tri-scFv includes a di-scFv linked to a third scFv domain including a Vk variable region (e.g., amino acids 539-649 of SEQ ID NO:40, or a functional variant or fragment thereof), linked to a VH variable domain (e.g., amino acids 671-786 of SEQ ID NO:40, or a functional variant or fragment thereof).

The Vk variable regions can be linked to VH variable domains by, for example, a linker (e.g., (GGGGS) 3 (SEQ ID NO:37), alone or in combination with a (6 aa) of light chain CH1 (amino acids 116-121 of SEQ ID NO: 39). Other suitable linkers are discussed above and known in the art. scFv can be linked by a linker (e.g., human IgG CH1 initial 13 amino acids (253-265) of SEQ ID NO:39), alone or in combination with a swivel sequence (e.g., amino acids 266-271 of SEQ ID NO:39). Other suitable linkers are discussed above and known in the art.

Therefore, a di-scFv can include amino acids 5-519 of SEQ ID NO: 39. A tri-scFv can include amino acids 5-786 of SEQ ID NO:40. In some embodiments, the fusion proteins include additional domains. For example, in some embodiments, the fusion proteins include sequences that enhance solubility (e.g., amino acids 1~4 of SEQ ID NO:39). Therefore, in some embodiments, a di-scFv can include amino acids 1-519 of SEQ ID NO: 39. A tri-scFv can include amino acids 1-786 of SEQ ID NO:40. In some embodiments that fusion proteins include one or more domains that enhance purification, isolation, capture, identification, separation, etc., of the fusion protein. Exemplary domains include, for example, Myc tag (e.g., amino acids 520-535 of SEQ ID NO:39) and/or a His tag (e.g., amino acids 536-541 of SEQ ID NO:39). Therefore, in some embodiments, a di-scFv can include the amino acid sequence of SEQ ID NO:39. A tri-scFv can include the amino acid sequence of SEQ ID NO:40. Other substitutable domains and additional domains are discussed in more detail above.

An exemplary 3E10 humanized Fv sequence is discussed in WO 2016/033324:

(SEQ ID NO: 41)
DIVLTQSPASLAVSPGQRATITCRASKSVSTSSYSYMHWYQQKPGQPPKLL
IYYASYLESGVPARFSGSGSGTDFTLTINPVEANDTANYYCQHSREFPWTF
GQGTKVEIKGGGGSGGGGSGGGGSEVQLVESGGGLVQPGGSLRLSCSASGF
TFSNYGMHWVRQAPGKGLEYVSYISSGSSTIYYADTVKGRFTISRDNSKNT
LYLQMSSLRAEDTAVYYCVKRGLLLDYWGQGTLVTVSS.

Exemplary 3E10 humanized di-scFv sequences are discussed in WO 2019/018426 and WO/2019/018428, and include:

(Variant 2, SEQ ID NO: 61)
DIQMTQSPSSLSASLGDRATITCRASKSVSTSSYSYMHWYQQKPGQPPKLL
IKYASYLESGVPSRFSGSGSGTDFTLTISSLQPEDAATYYCQHSREFPWTF
GGGTKVEIKRADAAPGGGGSGGGGSGGGGSEVQLVESGGGLVQPGGSLRLS
CAASGFTFSNYGMHWVRQAPGKGLEWVSYISSSSSTIYYADSVKGRFTISR
DNAKNSLYLQMNSLRAEDTAVYYCARRGLLLDYWGQGTTVTVSSASTKGPS
VFPLAPLESSGSDIQMTQSPSSLSASLGDRATITCRASKSVSTSSYSYMHW
YQQKPGQPPKLLIKYASYLESGVPSRFSGSGSGTDFTLTISSLQPEDAATY
YCQHSREFPWTFGGGTKVEIKRADAAPGGGGSGGGGSGGGGSEVQLVESGG
GLVQPGGSLRLSCAASGFTFSNYGMHWVRQAPGKGLEWVSYISSSSSTIYY
ADSVKGRFTISRDNAKNSLYLQMNSLRAEDTAVYYCARRGLLLDYWGQGTT
VTVSS,
(Variant 3, SEQ ID NO: 62)
DIQMTQSPSSLSASLGDRATITCRASKSVSTSSYSYMHWYQQKPGQPPKLL
IKYASYLESGVPSRFSGSGSGTDFTLTISSLQPEDAATYYCQHSREFPWTF
GGGTKVEIKRADAAPGGGGSGGGGSGGGGSEVQLVESGGGVVQPGGSLRLS
CAASGFTFSNYGMHWVRQAPEKGLEWVSYISSSSSTIYYADSVKGRFTISR
DNSKNTLYLQMNSLRAEDTAVYYCARRGLLLDYWGQGTTVTVSSASTKGPS
VFPLAPLESSGSDIQMTQSPSSLSASLGDRATITCRASKSVSTSSYSYMHW
YQQKPGQPPKLLIKYASYLESGVPSRFSGSGSGTDFTLTISSLQPEDAATY
YCQHSREFPWTFGGGTKVEIKRADAAPGGGGSGGGGSGGGGSEVQLVESGG
GVVQPGGSLRLSCAASGFTFSNYGMHWVRQAPEKGLEWVSYISSSSSTIYY
ADSVKGRFTISRDNSKNTLYLQMNSLRAEDTAVYYCARRGLLLDYWGQGTT
VTVSS,
(Variant 4, SEQ ID NO: 63)
DIQMTQSPSSLSASLGDRATITCRASKSVSTSSYSYMHWYQQKPGQPPKLL
IKYASYLESGVPSRFSGSGSGTDFTLTISSLQPEDAATYYCQHSREFPWTF
GGGTKVEIKRADAAPGGGGSGGGGSGGGGSEVQLVESGGGDVKPGGSLRLS
CAASGFTFSNYGMHWVRQAPEKGLEWVSYISSSSSTIYYADSVKGRFTISR
DNSKNTLYLQMNSLRAEDTAVYYCARRGLLLDYWGQGTTVTVSSASTKGPS
VFPLAPLESSGSDIQMTQSPSSLSASLGDRATITCRASKSVSTSSYSYMHW
YQQKPGQPPKLLIKYASYLESGVPSRFSGSGSGTDFTLTISSLQPEDAATY
YCQHSREFPWTFGGGTKVEIKRADAAPGGGGSGGGGSGGGGSEVQLVESGG
GDVKPGGSLRLSCAASGFTFSNYGMHWVRQAPEKGLEWVSYISSSSSTIYY
ADSVKGRFTISRDNSKNTLYLQMNSLRAEDTAVYYCARRGLLLDYWGQGTT
VTVSS,
(Variant 6, SEQ ID NO: 64)
DIQMTQSPSSLSASLGDRATITCRASKSVSTSSYSYMHWYQQKPGQAPKLL
IKYASYLESGVPSRFSGSGSGTDFTLTISSLQPEDFATYYCQHSREFPWTF
GQGTKVEIKRADAAPGGGGSGGGGSGGGGSEVQLVESGGGLVQPGGSLRLS
CAASGFTFSNYGMHWVRQAPGKGLEWVSYISSSSSTIYYADSVKGRFTISR
DNAKNSLYLQMNSLRAEDTAVYYCARRGLLLDYWGQGTTVTVSSASTKGPS
VFPLAPLESSGSDIQMTQSPSSLSASLGDRATITCRASKSVSTSSYSYMHW
YQQKPGQAPKLLIKYASYLESGVPSRFSGSGSGTDFTLTISSLQPEDFATY
YCQHSREFPWTFGQGTKVEIKRADAAPGGGGSGGGGSGGGGSEVQLVESGG
GLVQPGGSLRLSCAASGFTFSNYGMHWVRQAPGKGLEWVSYISSSSSTIYY
ADSVKGRFTISRDNAKNSLYLQMNSLRAEDTAVYYCARRGLLLDYWGQGTT
VTVSS,
(Variant 7, SEQ ID NO: 65)
DIQMTQSPSSLSASLGDRATITCRASKSVSTSSYSYMHWYQQKPGQAPKLL
IKYASYLESGVPSRFSGSGSGTDFTLTISSLQPEDFATYYCQHSREFPWTF
GQGTKVEIKRADAAPGGGGSGGGGSGGGGSEVQLVESGGGVVQPGGSLRLS
CAASGFTFSNYGMHWVRQAPEKGLEWVSYISSSSSTIYYADSVKGRFTISR
DNSKNTLYLQMNSLRAEDTAVYYCARRGLLLDYWGQGTTVTVSSASTKGPS
VFPLAPLESSGSDIQMTQSPSSLSASLGDRATITCRASKSVSTSSYSYMHW
YQQKPGQAPKLLIKYASYLESGVPSRFSGSGSGTDFTLTISSLQPEDFATY
YCQHSREFPWTFGQGTKVEIKRADAAPGGGGSGGGGSGGGGSEVQLVESGG
GVVQPGGSLRLSCAASGFTFSNYGMHWVRQAPEKGLEWVSYISSSSSTIYY
ADSVKGRFTISRDNSKNTLYLQMNSLRAEDTAVYYCARRGLLLDYWGQGTT
VTVSS,
(Variant 8, SEQ ID NO: 66)
DIQMTQSPSSLSASLGDRATITCRASKSVSTSSYSYMHWYQQKPGQAPKLL
IKYASYLESGVPSRFSGSGSGTDFTLTISSLQPEDFATYYCQHSREFPWTF
GQGTKVEIKRADAAPGGGGSGGGGSGGGGSEVQLVESGGGDVKPGGSLRLS
CAASGFTFSNYGMHWVRQAPEKGLEWVSYISSSSSTIYYADSVKGRFTISR
DNSKNTLYLQMNSLRAEDTAVYYCARRGLLLDYWGQGTTVTVSSASTKGPS
VFPLAPLESSGSDIQMTQSPSSLSASLGDRATITCRASKSVSTSSYSYMHW
YQQKPGQAPKLLIKYASYLESGVPSRFSGSGSGTDFTLTISSLQPEDFATY
YCQHSREFPWTFGQGTKVEIKRADAAPGGGGSGGGGSGGGGSEVQLVESGG
GDVKPGGSLRLSCAASGFTFSNYGMHWVRQAPEKGLEWVSYISSSSSTIYY
ADSVKGRFTISRDNSKNTLYLQMNSLRAEDTAVYYCARRGLLLDYWGQGTT
VTVSS,
(Variant 10, SEQ ID NO: 67)
DIQMTQSPSSLSASVGDRVTITCRASKSVSTSSYSYMHWYQQKPGKAPKLL
IKYASYLESGVPSRFSGSGSGTDFTLTISSLQPEDFATYYCQHSREFPWTF
GQGTKVEIKRADAAPGGGGSGGGGSGGGGSEVQLVESGGGLVQPGGSLRLS
CAASGFTFSNYGMHWVRQAPGKGLEWVSYISSSSSTIYYADSVKGRFTISR
DNAKNSLYLQMNSLRAEDTAVYYCARRGLLLDYWGQGTTVTVSSASTKGPS
VFPLAPLESSGSDIQMTQSPSSLSASVGDRVTITCRASKSVSTSSYSYMHW
YQQKPGKAPKLLIKYASYLESGVPSRFSGSGSGTDFTLTISSLQPEDFATY
YCQHSREFPWTFGQGTKVEIKRADAAPGGGGSGGGGSGGGGSEVQLVESGG
GLVQPGGSLRLSCAASGFTFSNYGMHWVRQAPGKGLEWVSYISSSSSTIYY
ADSVKGRFTISRDNAKNSLYLQMNSLRAEDTAVYYCARRGLLLDYWGQGTT
VTVSS,
(Variant 11, SEQ ID NO: 68)
DIQMTQSPSSLSASVGDRVTITCRASKSVSTSSYSYMHWYQQKPGKAPKLL
IKYASYLESGVPSRFSGSGSGTDFTLTISSLQPEDFATYYCQHSREFPWTF
GQGTKVEIKRADAAPGGGGSGGGGSGGGGSEVQLVESGGGVVQPGGSLRLS
CAASGFTFSNYGMHWVRQAPEKGLEWVSYISSSSSTIYYADSVKGRFTISR
DNSKNTLYLQMNSLRAEDTAVYYCARRGLLLDYWGQGTTVTVSSASTKGPS
VFPLAPLESSGSDIQMTQSPSSLSASVGDRVTITCRASKSVSTSSYSYMHW
YQQKPGKAPKLLIKYASYLESGVPSRFSGSGSGTDFTLTISSLQPEDFATY
YCQHSREFPWTFGQGTKVEIKRADAAPGGGGSGGGGSGGGGSEVQLVESGG
GVVQPGGSLRLSCAASGFTFSNYGMHWVRQAPEKGLEWVSYISSSSSTIYY
ADSVKGRFTISRDNSKNTLYLQMNSLRAEDTAVYYCARRGLLLDYWGQGTT
VTVSS,
(Variant 12, SEQ ID NO: 69)
DIQMTQSPSSLSASVGDRVTITCRASKSVSTSSYSYMHWYQQKPGKAPKLL
IKYASYLESGVPSRFSGSGSGTDFTLTISSLQPEDFATYYCQHSREFPWTF
GQGTKVEIKRADAAPGGGGSGGGGSGGGGSEVQLVESGGGDVKPGGSLRLS
CAASGFTFSNYGMHWVRQAPEKGLEWVSYISSSSSTIYYADSVKGRFTISR
DNSKNTLYLQMNSLRAEDTAVYYCARRGLLLDYWGQGTTVTVSSASTKGPS
VFPLAPLESSGSDIQMTQSPSSLSASVGDRVTITCRASKSVSTSSYSYMHW
YQQKPGKAPKLLIKYASYLESGVPSRFSGSGSGTDFTLTISSLQPEDFATY
YCQHSREFPWTFGQGTKVEIKRADAAPGGGGSGGGGSGGGGSEVQLVESGG
GDVKPGGSLRLSCAASGFTFSNYGMHWVRQAPEKGLEWVSYISSSSSTIYY
ADSVKGRFTISRDNSKNTLYLQMNSLRAEDTAVYYCARRGLLLDYWGQGTT
VTVSS,
(Variant 13, SEQ ID NO: 70)
DIQMTQSPSSLSASLGDRATITCRASKTVSTSSYSYMHWYQQKPGQPPKLL
IKYASYLESGVPSRFSGSGSGTDFTLTISSLQPEDAATYYCQHSREFPWTF
GGGTKVEIKRADAAPGGGGSGGGGSGGGGSEVQLVESGGGLVQPGGSLRLS
CAASGFTFSNYGMHWVRQAPGKGLEWVSYISSGSSTIYYADSVKGRFTISR
DNAKNSLYLQMNSLRAEDTAVYYCARRGLLLDYWGQGTTVTVSSASTKGPS
VFPLAPLESSGSDIQMTQSPSSLSASLGDRATITCRASKTVSTSSYSYMHW
YQQKPGQPPKLLIKYASYLESGVPSRFSGSGSGTDFTLTISSLQPEDAATY
YCQHSREFPWTFGGGTKVEIKRADAAPGGGGSGGGGSGGGGSEVQLVESGG
GLVQPGGSLRLSCAASGFTFSNYGMHWVRQAPGKGLEWVSYISSGSSTIYY
ADSVKGRFTISRDNAKNSLYLQMNSLRAEDTAVYYCARRGLLLDYWGQGTT
VTVSS,
(Variant 14, SEQ ID NO: 71)
DIQMTQSPSSLSASLGDRATITCRASKTVSTSSYSYMHWYQQKPGQPPKLL
IKYASYLESGVPSRFSGSGSGTDFTLTISSLQPEDAATYYCQHSREFPWTF
GGGTKVEIKRADAAPGGGGSGGGGSGGGGSEVQLVESGGGVVQPGGSLRLS
CAASGFTFSNYGMHWVRQAPEKGLEWVSYISSGSSTIYYADSVKGRFTISR
DNSKNTLYLQMNSLRAEDTAVYYCARRGLLLDYWGQGTTVTVSSASTKGPS
VFPLAPLESSGSDIQMTQSPSSLSASLGDRATITCRASKTVSTSSYSYMHW
YQQKPGQPPKLLIKYASYLESGVPSRFSGSGSGTDFTLTISSLQPEDAATY
YCQHSREFPWIFGGGTKVEIKRADAAPGGGGSGGGGSGGGGSEVQLVESGG
GVVQPGGSLRLSCAASGFTFSNYGMHWVRQAPEKGLEWVSYISSGSSTIYY
ADSVKGRFTISRDNSKNTLYLQMNSLRAEDTAVYYCARRGLLLDYWGQGTT
VTVSS,
(Variant 15, SEQ ID NO: 72)
DIQMTQSPSSLSASLGDRATITCRASKTVSTSSYSYMHWYQQKPGQPPKLL
IKYASYLESGVPSRFSGSGSGTDFTLTISSLQPEDAATYYCQHSREFPWTF
GGGTKVEIKRADAAPGGGGSGGGGSGGGGSEVQLVESGGGDVKPGGSLRLS
CAASGFTFSNYGMHWVRQAPEKGLEWVSYISSGSSTIYYADSVKGRFTISR
DNSKNTLYLQMNSLRAEDTAVYYCARRGLLLDYWGQGTTVTVSSASTKGPS
VFPLAPLESSGSDIQMTQSPSSLSASLGDRATITCRASKTVSTSSYSYMHW
YQQKPGQPPKLLIKYASYLESGVPSRFSGSGSGTDFTLTISSLQPEDAATY
YCQHSREFPWTFGGGTKVEIKRADAAPGGGGSGGGGSGGGGSEVQLVESGG
GDVKPGGSLRLSCAASGFTFSNYGMHWVRQAPEKGLEWVSYISSGSSTIYY
ADSVKGRFTISRDNSKNTLYLQMNSLRAEDTAVYYCARRGLLLDYWGQGTT
VTVSS,
(Variant 16, SEQ ID NO: 73)
DIQMTQSPSSLSASVGDRVTITCRASKTVSTSSYSYMHWYQQKPGKAPKLL
IKYASYLESGVPSRFSGSGSGTDFTLTISSLQPEDFATYYCQHSREFPWTF
GQGTKVEIKRADAAPGGGGSGGGGSGGGGSEVQLVESGGGLVQPGGSLRLS
CAASGFTFSNYGMHWVRQAPGKGLEWVSYISSGSSTIYYADSVKGRFTISR
DNAKNSLYLQMNSLRAEDTAVYYCARRGLLLDYWGQGTTVTVSSASTKGPS
VFPLAPLESSGSDIQMTQSPSSLSASVGDRVTITCRASKTVSTSSYSYMHW
YQQKPGKAPKLLIKYASYLESGVPSRFSGSGSGTDFTLTISSLQPEDFATY
YCQHSREFPWTFGQGTKVEIKRADAAPGGGGSGGGGSGGGGSEVQLVESGG
GLVQPGGSLRLSCAASGFTFSNYGMHWVRQAPGKGLEWVSYISSGSSTIYY
ADSVKGRFTISRDNAKNSLYLQMNSLRAEDTAVYYCARRGLLLDYWGQGTT
VTVSS,
(Variant 17, SEQ ID NO: 74)
DIQMTQSPSSLSASVGDRVTITCRASKTVSTSSYSYMHWYQQKPGKAPKLL
IKYASYLESGVPSRFSGSGSGTDFTLTISSLQPEDFATYYCQHSREFPWTF
GQGTKVEIKRADAAPGGGGSGGGGSGGGGSEVQLVESGGGVVQPGGSLRLS
CAASGFTFSNYGMHWVRQAPEKGLEWVSYISSGSSTIYYADSVKGRFTISR
DNSKNTLYLQMNSLRAEDTAVYYCARRGLLLDYWGQGTTVTVSSASTKGPS
VFPLAPLESSGSDIQMTQSPSSLSASVGDRVTITCRASKTVSTSSYSYMHW
YQQKPGKAPKLLIKYASYLESGVPSRFSGSGSGTDFTLTISSLQPEDFATY
YCQHSREFPWTFGQGTKVEIKRADAAPGGGGSGGGGSGGGGSEVQLVESGG
GVVQPGGSLRLSCAASGFTFSNYGMHWVRQAPEKGLEWVSYISSGSSTIYY
ADSVKGRFTISRDNSKNTLYLQMNSLRAEDTAVYYCARRGLLLDYWGQGTT
VTVSS,
(Variant 18, SEQ ID NO: 75)
DIQMTQSPSSLSASVGDRVTITCRASKTVSTSSYSYMHWYQQKPGKAPKLL
IKYASYLESGVPSRFSGSGSGTDFTLTISSLQPEDFATYYCQHSREFPWTF
GQGTKVEIKRADAAPGGGGSGGGGSGGGGSEVQLVESGGGDVKPGGSLRLS
CAASGFTFSNYGMHWVRQAPEKGLEWVSYISSGSSTIYYADSVKGRFTISR
DNSKNTLYLQMNSLRAEDTAVYYCARRGLLLDYWGQGTTVTVSSASTKGPS
VFPLAPLESSGSDIQMTQSPSSLSASVGDRVTITCRASKTVSTSSYSYMHW
YQQKPGKAPKLLIKYASYLESGVPSRFSGSGSGTDFTLTISSLQPEDFATY
YCQHSREFPWTFGQGTKVEIKRADAAPGGGGSGGGGSGGGGSEVQLVESGG
GDVKPGGSLRLSCAASGFTFSNYGMHWVRQAPEKGLEWVSYISSGSSTIYY
ADSVKGRFTISRDNSKNTLYLQMNSLRAEDTAVYYCARRGLLLDYWGQGTT
VTVSS,
and
(Variant 19, SEQ ID NO: 76)
DIQMTQSPSSLSASLGDRATITCRASKTVSTSSYSYMHWYQQKPGQAPKLL
IKYASYLESGVPSRFSGSGSGTDFTLTISSLQPEDFATYYCQHSREFPWTF
GQGTKVEIKRADAAPGGGGSGGGGSGGGGSEVQLVESGGGLVQPGGSLRLS
CAASGFTFSNYGMHWVRQAPGKGLEWVSYISSGSSTIYYADSVKGRFTISR
DNAKNSLYLQMNSLRAEDTAVYYCARRGLLLDYWGQGTTVTVSSASTKGPS
VFPLAPLESSGSDIQMTQSPSSLSASLGDRATITCRASKTVSTSSYSYMHW
YQQKPGQAPKLLIKYASYLESGVPSRFSGSGSGTDFTLTISSLQPEDFATY
YCQHSREFPWTFGQGTKVEIKRADAAPGGGGSGGGGSGGGGSEVQLVESGG
GLVQPGGSLRLSCAASGFTFSNYGMHWVRQAPGKGLEWVSYISSGSSTIYY
ADSVKGRFTISRDNAKNSLYLQMNSLRAEDTAVYYCARRGLLLDYWGQGTT
VTVSS.

### 3. Additional Sequences

Additional sequences that may used in the construction of cell-penetrating antigen binding proteins, antibodies, fragments and fusion proteins include, but are not limited to,

(IgG1 L2345A/L235A heavy chain full length sequence, SEQ ID NO: 77)
EVQLVESGGGLVQPGGSLRLSCAASGFTFSNYGMHWVRQAPGKGLEWVSYI
SSGSSTIYYADSVKGRFTISRDNAKNSLYLQMNSLRAEDTAVYYCARRGLL
LDYWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNHK
PSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSK
LTVDKSRWQQGNVFSCSVMHEALHNHYTQKSLSLSPGK,
(IgG1 constant heavy region 1, SEQ ID NO: 78)
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKV,
(IgG1 hinge region, SEQ ID NO: 79)
EPKSCDKTHTCP,
(IgG1 L2345A/L235A constant heavy region 2, SEQ ID NO: 80)
PCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALP
APIEKTISKAK,
(IgG1 constant heavy region 3, SEQ ID NO: 81)
GQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLS
LSPGK,
(IgG1 N297D heavy chain full length sequence, SEQ ID NO: 82)
EVQLVESGGGLVQPGGSLRLSCAASGFTFSNYGMHWVRQAPGKGLEWVSYI
SSGSSTIYYADSVKGRFTISRDNAKNSLYLQMNSLRAEDTAVYYCARRGLL
LDYWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNHK
PSNTKVDKKVEPKSCDKTHTCPPCPAPELLGGPSVFLFPPKPKDTLMISRT
PEVTCVVVDVSHEDPEVKFNWYVDGVEVHNAKTKPREEQYDSTYRVVSVLT
VLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSK
LTVDKSRWQQGNVFSCSVMHEALHNHYTQKSLSLSPGK,
(IgG1 N297D constant heavy region 2, SEQ ID NO: 83)
PCPAPELLGGPSVFLFPPKPKDTLMISRTPEVTCVVVDVSHEDPEVKFNWY
VDGVEVHNAKTKPREEQYDSTYRVVSVLTVLHQDWLNGKEYKCKVSNKALP
APIEKTISKAK,
(IgG1 L2345A/L235A/N297D heavy chain full length sequence, SEQ ID NO: 84)
EVQLVESGGGLVQPGGSLRLSCAASGFTFSNYGMHWVRQAPGKGLEWVSYI
SSGSSTIYYADSVKGRFTISRDNAKNSLYLQMNSLRAEDTAVYYCARRGLL
LDYWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNHK
PSNTKVDKKVEPKSCDKTHTCPPCPAPEAAGGPSVFLFPPKPKDTLMISRT
PEVTCVVVDVSHEDPEVKFNWYVDGVEVHNAKTKPREEQYDSTYRVVSVLT
VLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSK
LTVDKSRWQQGNVFSCSVMHEALHNHYTQKSLSLSPGK,
(IgG1 L2345A/L235A/N297D constant heavy region 2, SEQ ID NO: 85)
PCPAPEAAGGPSVFLFPPKPKDTLMISRTPEVTCVVVDVSHEDPEVKFNWY
VDGVEVHNAKTKPREEQYDSTYRVVSVLTVLHQDWLNGKEYKCKVSNKALP
APIEKTISKAK,
(SEQ ID NO: 86, Unmodified constant heavy region 2)
PCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALP
APIEKTISKAK,
(SEQ ID NO: 121, heavy chain constant region)
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHED
PEVKFNWYVDGVEVHNAKTKPREEQYNSTYRVVSVLIVLHQDWLNGKEYKC
KVSNKALPAPIEKTISKAKGQPREPQVYTLPPSRDELTKNQVSLWCLVKGF
YPSDIAVEWESNGQPENNYKTTPPVLDSDGSFFLYSKLTVDKSRWQQGNVF
SCSVMHEALHNHYTQKSLSLSPGK,
(SEQ ID NO: 122, heavy chain constant region)
ASVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSF
NRGECDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLSCA
VKGFYPSDIAVEWESNGQPENNYKTTPPVLDSDGSFFLVSKLTVDKSRWQQ
GNVFSCSVLHEALHNRFTQKSLSLSPGK,
(Light chain full length sequence, SEQ ID NO: 87)
DIQMTQSPSSLSASLGDRATITCRASKTVSTSSYSYMHWYQQKPGQPPKLL
IKYASYLESGVPSRFSGSGSGTDFTLTISSLQPEDAATYYCQHSREFPWTF
GGGTKLEIKRTVAAPSVFIFPPSDEQLKSGTASVVCLLNNFYPREAKVQWK
VDNALQSGNSQESVTEQDSKDSTYSLSSTLILSKADYEKHKVYACEVTHQG
LSSPVTKSFNRGEC.
(SEQ ID NO: 123, Light chain constant region)
RTVAAPSVFIFPPSDEQLKSGTASVVCLLNNFYPREAKVQWKVDNALQSGN
SQESVTEQDSKDSTYSLSSTLILSKADYEKHKVYACEVTHQGLSSPVTKSF
NRGEC,
and
(SEQ ID NO: 124, Light chain constant region)
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKS
C.

### IV. Targets & Exemplary Antibodies for Proteolysis of Target Proteins

Examples of antibodies from which the antigen binding feature for binding to protein(s) targeted for proteolysis can be derived are also provided. Such antibodies can be, for example, commercially available antibodies or other antibodies for which the antigen binding domain (e.g., CDRs, heavy and light chain variable domains, etc.) is known.

In some embodiments, the antibodies target, for example, DNA repair and/or DNA damage response factors, oncogenes like RAS and MYC, anti-apoptotic/pro-survival factors such as BCL-2pro, growth and transcription factors such as androgen receptor, double homeobox 4 also known as DUX4 (misexpression is the cause of facioscapulohumeral muscular dystrophy (FSHD)), mutant huntingtin protein, amyloid protein, tau protein, etc.

Specific exemplary target proteins include, but are not limited to, 4E-BP1, 5-hydroxyuridine, A1BG, AAT, ABCB1, ABCB1, ABCB5, ABCC4, ABCG2, ABCG5, ABL1, ABL2, ACLY, ACP5, ACSS1, ACTA2, ACTH, ACTR3, ACVR1, ADAM10, ADAMTS1, ADAP1, ADAR, ADD1, ADIPOQ, ADORA2A, ADRB2, AEBP2, AFAP1L2, AFP, AGR2, AGT, AHR, AIB1, AIF, AIM2, AKT1, AKT1S1, AKT2, Akt3, AKT3, ALB, ALCAM, AlCAM, ALCAM, ALDH1A1, ALDH2, ALDH6A1, ALDOA, ALK,ALK/p80, ALPI, ALPL, ALPP, AMACR, AMBRA1, AMY1A, ANAPC1, ANAPC10, ANAPC11, Androgen receptor, ANPEP, ANXA5, AOF1, AP2M1, APAF1, APBA2, APBB1IP, APC2, APEX1, APOA1, APOA4, APOA5, Apoa5 (ab), Apoa5 (bd), ApoB, ApoE, APOL1, ApoM, ApoM, APP, AQP2, AR, ARF1, ARFGAP1, ARG1, ARHGAP42, ARHGDIA, ARNTL, ASFIA, ASFIB, ASGR2, ASH2L, ASS1, ATF2, ATF3, ATF4, ATG10, ATG13, ATG14L, ATG16L1, ATG2A, ATG3, ATG4A, ATG4B, ATG4C, ATG5, ATG7, ATL1, ATM, ATP2A1, ATP2C1, ATPIF1, ATR, ATRX, ATXN1, AUP1, AURKA, AURKA, AURKB, AXINI, AXL, B2M, B3GAT1, B7H3, B7H4, BACEI, BAD, BAGI, BAKI, BATF, BAX, BCAT2, BCL10, BCL11B, BCL-2, BCL2L10, BCL2L2, BCL6, BCL9L, BCR, BDH1, BDNF, BECN1, beta Amyloid, beta-Actin, BHMT, BID, BIN1, BIRC5, BLK, BLNK, BMI1, BMP2, BMP4, BMP7, BMPR1A, BMPR2, BMX, BNIP3, BNIP3L, BNP, BNP, Bpifa2, BPTF, BRAF, BRCA1, BRD2, BRIP1, BSA, BTK, BTLA, BTN1A1, BTN2A2, BTN3A1, BTRC, BUB1, C17ORF53, C1QA, C1QC, C3C, CA1, CA9, CALB2, Calcyclin, CALD1, Calnexin, CALR, Calreticulin, CAMK2G, CAMK4, CAMP, CAPN1, CAPN2, CARM1, CASP3, CASP6, CASP-7, CASP8, CASP9, CAV2, CBX1, CBX2, CBX3, CBX4, CBX5, CBX6, CBX7, CBX8, C-CBL, CCL2, CCL4, CCNA2, CCNB1, CCNB1, CCND1, CCND1, CCNE1, CCT2, CCT2, CD10, CD100, CD101, CD102, CD102, CD104, CD104, CD105, CD106, CD107b, CD108, CD109, CD110, CD112, CD113, CD114, CD118, CD119, CD11a, CD11C, CD11D, CD120B, CD122, CD124, CD125, CD127, CD129, CD130, CD131, CD132, CD133, CD134, CD137, CD14, CD140a, CD141, CD143, CD147, CD148, CD15, CD152, CD154, CD155, CD156, CD158D, CD158E1, CD16, CD161, CD163, CD163, CD166, CD167, CD168, CD169, CD16B, CD170, CD171, CD172A, CD172G, CD174, CD177, CD179A, CD18, CD181, CD182, CD183, CD19, CD191, CD192, CD193, CD195, CD196, CD197, CD1A, CD1B, CD1C, CD2, CD20, CD200, CD201, CD202B, CD203C, CD204, CD205, CD207, CD208, CD209, CD21, CD213A1, CD215, CD217, CD218A, CD22, CD222, CD223, CD224, CD226, CD227, CD230, CD232, CD239, CD24, CD243, CD247, CD248, CD256, CD267, CD268, CD269, CD27, CD274, CD275, CD276, CD282, CD283, CD289, CD299, CD3, CD30, CD300A, CD300C, CD301, CD302, CD303, CD305, CD306, CD307A, CD307B, CD307C, CD307E, CD31, CD314, CD315, CD319, CD322, CD327, CD328, CD329, CD33, CD334, CD334, CD335, CD336, CD339, CD34, CD344, CD351, CD352, CD353, CD354, CD355, CD36, CD360, CD361, CD363, CD364, CD367, CD368, CD369, CD37, CD370, CD38, CD39, CD3D, CD3E, CD3G, CD4, CD40, CD42A, CD42B, CD43, CD44, CD44, CD45, CD45R, CD45R, CD46, CD48, CD49B, CD49C, CD49D, CD49E, CD5, CD50, CD53, CD53, CD54, CD57, CD59, CD6, CD61, CD63, CD64, CD66A, CD66B, CD68, CD69, CD7, CD72, CD74, CD79B, CD8, CD80, CD83, CD84, CD85G, CD86, CD87, CD89, CD89, CD8A, CD9, CD93, CD94, CD96, CD97, CD99, CDC2, CDC25C, CDC27, CDC37, CDCP1, CDH1, CDH11, CDH17, CDH2, CDH5, CDK1, CDK2, CDK5, CDK9, CDKN1B, CDKNIC, CDKN2A, CDKN2A/P16, CDX1, CDX2, CEA, CEACAM5, CEBPA, CEBPB, CER1, CFHR5, CFLAR, CGA, CGB, CHD3, CHD4, CHGA, CHIT1, CHK2, CHRM3, CHRM5, CHRNA10, CHRNA2, CHRNA3, CHRNA4, CHRNA5, CHRNA6, CHRNA7, CHRNB2, CHRNB3, CHRNB4, CHRND, CHRNE, CHUK, CIB1, CIDEC, CIRBP, c-Jun, CK1, CK17, CK5, CK7, C-Kit, CKM, CLDN6, Clenbuterol, CLGN, CLL1, CLOCK, C-MYC, CNN1, CNN3, COL1A1, COL1A2, COL3A1, COTL1, COX411, CPV-NS1, CPV-VP2, CREB1, c-Rel, CRK, CRKL, CRP, CRTC1, CRTC2, CRTC3, CRYAB, CSF1, CSF1R, CSF2, CSF3, CSK, CSNK2A2, CSNK2B, CSPG4, CST3, CTCF, cTnI, CTNNA1, CTNNB1, CTNNBL1, CTSD, CTTN, CXCL16, CYCS, CYLD, CYP1A1, CYP3A4, Cytokeratin (Pan), Cytokeratin 18, Cytokeratin 19, Cytokeratin 5, Cytokeratin 5, Cytokeratin 8, Cytokeratin (Pan), DAPK3, DAPP1, DAXX, DCN, DCN, DCTN4, DCX, DDR1, DDR2, DDX1, DDX20, DDX39B, DDX3X, DDX4, DDX5, DDX58, deltaNp63alpha, Desmin, DFFA, DFFB, DHX58, DIS3L2, DKK1, DKK3, DLK1, DLL4, DNAL4, DNMIL, DNMT1, DNMT3A, DNMT3B, DNMT3L, DNTT, DOC2, DSG3, DUX4, Dynamin-1, Dynamin-2, E2F1, E7, EDA2R, EEF2, EGF, EGFR, EGFR mutant, EGR1, EHMT2, EhpB1, EhpB6, EIF2A, EIF2AK2, EIF2AK3, EIF4B, EIF4E, EIF5, EIF5A, ELANE, ELK1, EMD, ENO2, eNOS, EP300, EPCAM, EphA1, Epha10, EphA2, EphA3, EphA4, EphA5, EphA6, EphA7, EphA8, EphB2, EphB3, EphB4, EphB4, EphB6, EPN1, EPO, ER, ER-alpha, ERBB2, ERBB3, ERBB3, ERBB4, ERCC1, ERK2, ERK3, ERN1, ESR1, ESRRA, ETS1, ETV1, ETV4, ETV5, EZH1, EZH2, EZR, F2R, F8, FABP2, FABP4, FADD, FAK, FAP, FAS, FBLN2, FBLN5, FBXL10, FCER1A, FCER2, FCGRT, FER, FES, FGB, FGF2, FGF4, FGFR1, FGFR3, FGFR4, FGG, FGL1, FGR, Fibulin 5, Flag, FLI1, FLT1, FLT3, FLT4, FMR1, FN1, FOLR1, FOS, FOXA2, FOXC2, FOXD3, FOXD3, FOXM1, FOXO1, FOXP1, FOXP2, FOXP3, FRK, FSHR, FTL, FUK, FUT4, FYN, FZD5, G6PD, GAB1, GABBR2, GABPA, GAD2, GAPDH, GATA1, GATA3, GATA4, GATA5, GATA6, GCG, GCK, GFAP, GFI1, GFP, GFPT1, GHI, GITR, GKAP, GLI1, GLP, GLRA1, GLRB, Glucose-6-phosphate isomerase, GLUL, GNAS, GNL3, GOT2, GPC3, GPNMB, GRIA2, GRIA3, GRIK2, GRIK3, GRIK4, GRIK5, GRIN2A, GRIN2B, GRIN3B, GRK2, GRM1, GRM2, GRM3, GRM5, GRM6, GRM7, GRM8, GSC, GSK3 alpha, GSK3B, GSN, GST, GSTM1, GSTP1, GUCY1A3, GYS1, GZMB, HAND1, HAS1, HAS2, HAS3, HAUSP, HAVCR1, HCK, HDAC10, HDAC2, HDAC3, HDAC4, HDAC6, HDAC9, HER-2, HEXA, HFE, HH3, HIFIA, HIST2H3C (27Ac), HIST2H4A (20Me), HIST2H4A (20Me), HIST2H4A (20Me3), HK1, HK2, HLA-B, HLA-DRA, HOXA9, HOXB4, HPRT, HPRT1, HPS1, HRP, HSF1, HSF4, HSP27, HSP60, HSP70, HSP90AA1, HSP90AB1, HSP90AB1, HSPA5, HSPB2, HTR3A, HTR3B, HTRA2, human Albumin, human IgG, human IgG (Fc Specific), human IgM, Human P16, human Splunc2, Huntingtin, ICAM1, ID2, IDH1, IDH2, IFN-gamma, IGFIR-Beta, IGF2, IGF2BP3, IGFBP2, IGFBP7, IghA1, IGHM, IGLC2, IHOG, IKBKB, IKBKE, IL-10, IL10RB, IL-16, IL-1a, IL1B, IL1R1, IL1RAPL1, IL-2, IL28A, IL2RA, IL34, IL3RA, IL-6, IL-8, ILK, INCENP, Influenza A virus Nucleoprotein, Influenza B virus Nucleoprotein, INHA (Inhibin alpha), IRAK3, IRAK4, ISL1, ITGA2B, ITGA4, ITGA5, ITGAM, ITGB1, ITGB4, ITK, JAK2, JAK3, JARID2, JUP, KARS, KAT2B, KAT6B, KAT7, KAT7, KBTBD8, KCND2, KCNQ1, KDM1A, KDM2A, KDM3A, KDM4A, KDM4B, KDM5B, KDM6A, KDR, KEAP1, KHDRBS2, Ki67, KID, KIR3DL1, KIT, KLF1, KLF15, KLF2, KLF4, KLF6, KLHL1, KLHL11, KLHL12, KLHL13, KLHL21, KLHL22, KLHL25, KLK3, KMT2A, KMT2C, KMT2D, KMT5A, KPNA2, KRT10, KRT13, KRT15, KRT18, KRT19, KRT20, KSHV K8a, KSHV ORF26, KSHV ORF45, KSHV ORF62, KSHV ORF8, LAL, LAMB1, Lck, LCN1, LDLR, LEF1, LGALS1, LGR5, LHCGR, LHX2, LILRA1, LILRA2, LILRA3, LILRA5, LILRA6, LILRB1, LILRB2, LILRB5, LIN28, LMNA, LMNB2, LMO2, LPA, LPL, LPlunc1, LPP, LRP1, LRP12, LRP1B, LRP3, LRP4, LRP5, LRP6, LRP8, LSD1/AOF2, LTBR, LYN, Mammaglobin-1, MAP1LC3A, MAP1LC3B, MAP2, MAP2K2, MAP2K3, MAP2K4, MAP2K6, MAP2K7, MAP3K14, MAP3K2, MAP3K5, MAP3K7, MAP4K4, MAP4K4, MAPK10, MAPK11, MAPK14, MAPK3, MAPK8, MAPK9, MARK3, MATK, MATN1, MB, MBP, MCAM, MCL-1, MCM2, MCM3AP, MCP-1, MDM2, MDM4, MECP2, MEF2A, MEF2C, MELK, MEN1, MER, MESP1, MESP2, MET, Metadherin, MGMT, MIB1, MITF, MLANA, MLH1, MLL, MLXIPL, MMEL1, MMP1, MMP14, MMP2, MMP3, MMP9, MOB1A, mouse Lplunc1, mouse Splunc2, Mouse TUG, MPL, MPS1, MRPL42, MSH2, MSH6, MSI1, MSI2, MSI2, MSLN, MSN, MSTN, MSX1, MTA1, MTHFR, mTOR, MUC16, MUC2, MUC5AC, MUC5B, MUM1, MuRF1, MUSK, MYC, MYCN, Myeloperoxidase, MYF6, MYL2, MYL3, MYLK, MYOD1, Myoglobin, Myostatin, MYST1, NAA10, NACC1, NAGR1, NANOG, NAPSA, NBN, NCAM1, NCK1, NCOA3, NCOR1, NEDD8, NEFH, NEFL, NEFL, NEFM, NES, Neurod, NeuroD, Neurod, Neuropilin-1, NFE2L2, NFKB1, NFKB2, NFKBIA, NFKBIB, NF-κB p65, NGFR, NKX2.2, NKX2.5, NKX3A, NME1, NME2, NOS1, NOS2, Notch3, Notch3, NOX4, NPC1, NPT, NQO1, NR112, NR2C2, NR3C1, NR6A1, NRCAM, NRP1, NS1, NT5E, NTRK2, NTRK3, Nucleophosmin, Oct4, OLIG2, ONECUT3, OTUD4, OTX2, OVA, OX40, P16 (Mouse and Human), P2RX7, P2RY1, P2RY1, P2RY12, P2RY13, P2RY14, P2RY2, P2RY4, P2RY8, p44/42 MAPK (Erk1/2), p53, p63a, PAK2, PAK3, PAPLN, PAR1, PAR4, PARL, PARP, PAX2, PAX3, PAX4, PAX5, PAX6, PAX8, PBEF1, PBK, PCDH9, PCK2, PCNA, PCT, PD1, PDE1B, PDGFRA, PDGFRB, PDGFRB, PDK1, PDK2, PDPK1, PDX1, PEG10, PELP1, PER3, PGA5, PGC-1alpha, PGR, PGRMC1, PHB, PHC1, Phospho-4E-BP1 (Ser65), phospho-Eralpha (Tyr-537), phospho-NLRC4 (Ser-533), PIDD1, PIGR, PIK3CA, PIK3CG, PIK3R1, PIK3R4, PINCH, PINK1, Pirh2, PIWIL4, PKHD1, PKN1, PLA2G12A, PLA2G7, PLAGLI, PLCG1, PLCG2, PLD2, PLIN2, PLK1, PLXNA1, PMAIP1, PMS2, PODXL, POLR2A, POMC, PON1, POU3F2, PPARA, PPARG, PPARGC1B, PPMIA, PPP1A, PPP1CA, PPP1CB, PPP1R1B, PPP2R4, PPY, PR, PRAK, PRDM1, PRDM14, PRDM4, PRDM4, PRDM5, PRK2, PRKAA1, PRKAA2, PRKAB2, PRKACA, PRKACG, PRKAG1, PRKAG3, PRKDC, PRKN, PRMT6, PROM1, Proteinase 3, PROZ, PSA (KLK3), PSAP, PSG1, PSIP1, PSMA, PSMB8, PSMC3, PTEN, PTH, PTHIR, PTK6, PTK7, PTP4A2, PTPN1, PTPN11, PTPN14, PTPN6, PTPRC, PTPRD, PTPRF, PTPRM, PTPRU, PYCARD, PYK2, Rab10, RAB11FIP1, Rab13, Rab1b, Rab25, RAB27A, RAB27B, Rab3a, RAB4A, Rab5a, Rab6b, Rab8, Ractopamine, RAD18, RAD21, RAD23A, RAD50, RAD52, RAF1, RAG1, RAG2, RALA, RALB, RAN, RANGAP1, RAPIA, RAPIGAP, RAS, RB, RB1CC1, RBBP7, RBFOX2, RBFOX3, RBL2, RBP4, REGIA, RET, RF1, RHOF, RICTOR, Ring1, RIPK1, RIPK4, RND3, RNLS, ROCK1, RON, ROP1, ROR1, ROR2, RPA1, RPL18A, RPS6KA2, RPS6KB1, RPTOR, RRM1, RSK1, RSK2, R-spondin1, RTN3, RUBCN, RUNX1, RUNX3, S100A1, S100A10/P11, S100B, SAA1, SAG2, SALL4, SARS-COV-2, SARS-Cov2-NP1, SARS-Cov2-NP1, SARS-Cov2-NP2, SARS-COV-2-NP3, SARS-E2, SARS-M, SATB2, SATB2, SCGB1A1, SCGB2A2, SDC1, SDHB, SEC31A, SELL, SERPINA, SERPINA1, SERPINA3, SERPINA7, SERPINE1, SETD2, SETD7, SETDB1, SFTPC, SH3GL1, SHC1, SHH, SIRT1, SIRT2, SIRT3, SIRT4, SIRT6, SIRT7, SK2, SKP1, SKP2, SLC22A1, SLC27A2, SLC27A5, SLC2A4, SLINGSHOT-1L, SMAD1, SMAD2, SMAD3, SMAD4, SMAD5, SMAD6, SMARCA1, SMC1, SMCP, SMN1, SMYD2, SNAI1, SNAI2, SNCA, SNCG (breast cancer-specific protein 1), SND1/P100, SOD1, SOD2, SORL1, SOX10, SOX11, SOX2, SOX9, SP10, SP17, SPI1, SPIB, SPP1, SRA, SRA, SRC, SRY, SST, SSTR2, STAT3, STAT5A, STAT5B, STAT6, STK11, STYK1, Survivin, SUZ12, SYCP3, SYK, SYN1, SYN1, SYP, SYT1, T,TAB2, TARDBP, Tau, TBC1D4, TBCC, TBP, TBX5, TCF3, TCF4, TCL1A, TEC, TERF2, TERT, TET2, TFAP2A, TFAP2B, TFAP2C, TFF2, TFRC, TGF beta1, TGFb1, TGFBR3, TH, THAP1, THAP11, THBS1, Thioredoxin (TRX), THPO, THY1, TIA1, TIEI, TIGIT, TIM3, TIP60, TLL1, Tlr2, TLR9, TNF-alpha, TNFRSF10B, TNFRSF10D, TNFRSF11A, TNFRSF11B, TNFRSF12A, TNFRSF18, TNFRSF19, TNFRSF25, TNFRSF6B, TNFSF11, TNFSF13B, TNK1, TNNI2, TOP2A, TP53BP1, TRADD, TRAF2, TRAFD1, TRIM25, TRIM29, Trim5a, TRIP6, TrkA, troponin T2, TSHB, TSLPR, TTF1, TTR, TUBA4A, TUBA8, TUBB1, TUBB2A, TUBB3, TUBE1, TWF1, TWIST1, TWIST2, Tyk2, TYRO3, UBB, UBEIL, UBE2C, UBE2I, UCP2, UCP3, UFDIL, UHRF1, ULBP1, ULBP2, ULK2, UTF1, VAV1, VAV2, VCAMI, VEGFA, VIL1, VIM, Vimentin, VIMP, VISTA, VP2, VTN, WAS, WDFY3, WDR5, WDR66, WHSC2, WIFI, WNT1, WNT10B, WNT3A, WNT5A, WT1, WTAP, XBP1, XIAP, XRCC5, XRCC6, XRN2, YAP1, YES1, YWHAB, YWHAQ, ZAP70, ZBTB16, ZBTB7B, ZEB1, ZFP42, and ZFP91.

Antibodies that bind to each of the foregoing target proteins are known in the art and can be used in construction of the disclosed constructs and antibodies. For example, in some embodiments, the antigen binding feature includes the CDRs and optionally the heavy and light variable regions of an antibody that binds to one of foregoing proteins.

### V. Pharmaceutical Compositions

The compositions can be used therapeutically in combination with a pharmaceutically acceptable carrier.

The compositions including molecules for targeted proteolysis such as bispecific antibody are preferably employed for therapeutic uses in combination with a suitable pharmaceutical carrier. Such compositions include an effective amount of the composition, and a pharmaceutically acceptable carrier or excipient.

The compositions may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The molecules may also be encapsulated in suitable biocompatible particles formed of biodegradable or non-biodegradable polymers or proteins or liposomes for targeting to cells. Such systems are well known to those skilled in the art. In some embodiments, the molecules are encapsulated in nanoparticles.

Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). The materials may be in solution, emulsions, or suspension (for example, incorporated into particles, liposomes, or cells). Typically, an appropriate amount of a pharmaceutically-acceptable salt is used in the formulation to render the formulation isotonic. Trehalose, typically in the amount of 1-5%, may be added to the pharmaceutical compositions. The pH of the solution is preferably from about 5 to about 8, and more preferably from about 7 to about 7.5.

Pharmaceutical compositions may include carriers, thickeners, diluents, buffers, preservatives, and surface-active agents. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The compositions alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the include pharmaceutically acceptable carriers with formulation ingredients such as salts, carriers, buffering agents, emulsifiers, diluents, excipients, chelating agents, preservatives, solubilizers, or stabilizers.

Further carriers include sustained release preparations such as semi-permeable matrices of solid hydrophobic polymers containing the molecules, which matrices are in the form of shaped particles, e.g., films, liposomes or microparticles. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems. Inhalation includes administering the composition with an aerosol in an inhaler, either alone or attached to a carrier that can be absorbed. For systemic administration, it may be preferred that the composition is encapsulated in liposomes.

The compositions may be delivered in a manner which enables tissue-specific, using for example, invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed to result in sequential exposures to the composition, over a certain time period, for example, hours, days, weeks, months or years. This may be accomplished, for example, by repeated administrations of a formulation or by a sustained or controlled release delivery system in which the compositions are delivered over a prolonged period without repeated administrations.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulations.

Compositions can be formulated for pulmonary or mucosal administration. The administration can include delivery of the composition to the lungs, nasal, oral (sublingual, buccal), vaginal, or rectal mucosa. The term aerosol as used herein refers to any preparation of a fine mist of particles, which can be in solution or a suspension, whether or not it is produced using a propellant. Aerosols can be produced using standard techniques, such as ultrasonication or high-pressure treatment.

For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers.

The compositions can be delivered to the target cells using a particle delivery vehicle. Nanoparticles generally refers to particles in the range of between 500 nm to less than 0.5 nm, preferably having a diameter that is between 50 and 500 nm, more preferably having a diameter that is between 50 and 300 nm. Cellular internalization of polymeric particles is highly dependent upon their size, with nanoparticulate polymeric particles being internalized by cells with much higher efficiency than micoparticulate polymeric particles. For example, Desai, et al. have demonstrated that about 2.5 times more nanoparticles that are 100 nm in diameter are taken up by cultured Caco-2 cells as compared to microparticles having a diameter on 1 μM (Desai, et al., Pharm. Res., 14:1568-73 (1997)). Nanoparticles also have a greater ability to diffuse deeper into tissues in vivo.

In some embodiments, the delivery vehicle is a dendrimer.

Examples of preferred biodegradable polymers include synthetic polymers that degrade by hydrolysis such as poly(hydroxy acids), such as polymers and copolymers of lactic acid and glycolic acid, other degradable polyesters, polyanhydrides, poly(ortho) esters, polyesters, polyurethanes, poly(butic acid), poly(valeric acid), poly(caprolactone), poly(hydroxyalkanoates), poly(lactide-co-caprolactone), and poly(amine-co-ester) polymers, such as those described in Zhou, et al., Nature Materials, 11:82-90 (2012) and WO 2013/082529, U.S. Published Application No. 2014/0342003, and PCT/US2015/061375.

Targeting moieties can be associated with, linked, conjugated, or otherwise attached directly or indirectly to a nanoparticle or other delivery vehicle thereof. Targeting molecules can be proteins, peptides, nucleic acid molecules, saccharides or polysaccharides that bind to a receptor or other molecule on the surface of a targeted cell. The degree of specificity and the avidity of binding to the graft can be modulated through the selection of the targeting molecule.

Examples of moieties include, for example, targeting moieties which provide for the delivery of molecules to specific cells. Examples of molecules targeting extracellular matrix (“ECM”) include glycosaminoglycan (“GAG”) and collagen. In one embodiment, the external surface of polymer particles may be modified to enhance the ability of the particles to interact with selected cells or tissue. The method described above wherein an adaptor element conjugated to a targeting molecule is inserted into the particle is preferred. However, in another embodiment, the outer surface of a polymer micro- or nanoparticle having a carboxy terminus may be linked to targeting molecules that have a free amine terminus.

In another embodiment, the outer surface of the particle may be treated using a mannose amine, thereby mannosylating the outer surface of the particle. This treatment may cause the particle to bind to the target cell or tissue at a mannose receptor on the antigen presenting cell surface. Alternatively, surface conjugation with an immunoglobulin molecule containing an Fc portion (targeting Fc receptor), heat shock protein moiety (HSP receptor), phosphatidylserine (scavenger receptors), and lipopolysaccharide (LPS) are additional receptor targets on cells or tissue.

Lectins that can be covalently attached to micro- and nanoparticles to render them target specific to the mucin and mucosal cell layer.

The choice of targeting moiety will depend on the method of administration of the nanoparticle composition and the cells or tissues to be targeted. The targeting molecule may generally increase the binding affinity of the particles for cell or tissues or may target the nanoparticle to a particular tissue in an organ or a particular cell type in a tissue. In some embodiments, the targeting moiety targets the thymus, spleen, or cancer cells.

The covalent attachment of any of the natural components of mucin in either pure or partially purified form to the particles would decrease the surface tension of the bead-gut interface and increase the solubility of the bead in the mucin layer. The attachment of polyamino acids containing extra pendant carboxylic acid side groups, e.g., polyaspartic acid and polyglutamic acid, increases bioadhesiveness. Using polyamino acids in the 15,000 to 50,000 kDa molecular weight range yields chains of 120 to 425 amino acid residues attached to the surface of the particles. The polyamino chains increase bioadhesion by means of chain entanglement in mucin strands as well as by increased carboxylic charge.

### VI. Methods of Use

Methods of use are also provided. The methods typically include contacting cells with an effective amount of disclosed molecules to enhance proteolytic degradation of one or more target proteins through the TRIM21 pathway.

The molecules can be homogenous, e.g., all of the same type targeting the same protein for degradation. The molecules can also be heterogenous, e.g., of two or more different types targeting the same or different proteins for degradation.

Results show that the effectiveness of the TRIM21 pathway may be increased by inducing clustering of TRIM21 (Zeng, et al., “Target-induced clustering activates Trim-Away of pathogens and proteins”, Nat Struct Mol Biol 28, 278-289 (2021). doi.org/10.1038/s41594-021-00560-2. This may be accomplished by targeting multiple epitopes on the same target protein (e.g., using a multispecific molecule) and/or using two or more different molecules that target different epitopes on the same target protein.

Cell-penetration of certain antibodies such as 3E10 may be enhanced by the presence of extracellular nucleic acid. Thus, in some embodiments, the disclosed compositions are administered in combination with nucleic acids, in the same or separate admixture.

In some embodiments, the compositions are administered in an effective amount or therapeutically effective amount sufficient to treat, inhibit, or alleviate one or more symptoms of a disease or disorder, or to otherwise provide a desired pharmacologic and/or physiologic effect, for example, reducing, inhibiting, or reversing one or more of the pathophysiological mechanisms underlying a disease or disorder. In some embodiments, the protein targeted for degradation contributes to the underlying cause of the disease or disorder. For example, in some embodiments, the protein is overexpressed expressed, aberrantly expressed, and/or is a mutant relative to the wildtype protein. Exemplary target proteins are discussed above, any of which can be the subject(s) of the disclosed methods.

The formulation of the composition is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the molecules. The precise dosage will vary according to a variety of factors such as subject-dependent variables (e.g., age, immune system health, clinical symptoms etc.).

The composition can be administered or otherwise contacted with target cells once, twice, or three times daily; one, two, three, four, five, six, seven times a week, one, two, three, four, five, six, seven or eight times a month. For example, in some embodiments, the composition is administered every two or three days, or on average about 2 to about 4 times about week. Thus, in some embodiments, the composition is administered as part of dosage regimen including two or more separate treatments.

Dosage regimens include maintenance regimens, where the dosage remains the same between two or more administrations, escalation regimens where the dosage increases between two or more administrations, de-escalation regimens, where the dosage decreases between two or more administrations, or a combination thereof.

In some embodiments, the first dose can be a low dose. Dose escalation can be continued until a satisfactory biochemical or clinical response is reached. The clinical response will depend on the disease or disorder being treated, and/or the desired outcome. In some embodiments the dosage may increase until a therapeutic effect is identified, preferably without also inducing undesired toxicity or an acceptably high amount thereof. Next, the dosages can be maintained or steadily reduced to a maintenance dose. The methods can used to standardize, optimize, or customize the dose level, dose frequency, or duration of the therapy.

### A. In Vivo Methods

In some embodiments, in vivo treatment of a disease or disorder in a subject. The composition can be administered directly to a subject for in vivo therapy. In some embodiments, the bispecific antibody targets an antigen (e.g., protein) associated with the disease or disorder to be treated. For example, if the disease or disorder is caused by an over-expressed and/or mutant protein, the disclosed compositions and methods can be used to increase targeted degradation of the protein in an effective amount to treat the subject or the disease or disorder.

In general, methods of administering compounds, including antibodies, are well known in the art. The compositions can be administered by a number of routes including, but not limited to, intravenous, intraperitoneal, intraamniotic, intramuscular, subcutaneous, or topical (sublingual, rectal, intranasal, pulmonary, rectal mucosa, and vaginal), and oral (sublingual, buccal). In preferred embodiments, the composition is injected or infused into the animal.

In some embodiments, the composition is formulated for pulmonary delivery, such as intranasal administration or oral inhalation. Administration of the formulations may be accomplished by any acceptable method that allows the molecules to reach their targets. The administration may be localized (i.e., to a particular region, physiological system, tissue, organ, or cell type) or systemic, depending on the condition being treated. Compositions and methods for in vivo delivery are also discussed in WO 2017/143042.

The methods can also include administering an effective amount of the composition to an embryo or fetus, or the pregnant mother thereof, in vivo. In some methods, compositions are delivered in utero by injecting and/or infusing the compositions into a vein or artery, such as the vitelline vein or the umbilical vein, or into the amniotic sac of an embryo or fetus. See, e.g., Ricciardi, et al., Nat Commun. 2018 Jun. 26; 9 (1): 2481. doi: 10.1038/s41467-018-04894-2, and WO 2018/187493.

### B. In Vitro and Ex Vivo Methods

For in vitro and ex vivo methods, cells are typically contacted with the composition while in culture. For ex vivo methods, cells may be isolated from a subject and contacted ex vivo with the composition to produce cells containing the molecules. In a preferred embodiment, the cells are isolated from the subject to be treated or from a syngeneic host. Target cells can be removed from a subject prior to contacting with composition.

The disclosed invention can be further understood by the following numbered paragraphs.
1. An antigen binding molecule including (i) a TRIM21 binding feature, (ii) the cell-penetrating feature of a cell-penetrating antibody, and (iii) an antigen binding feature that binds to a target protein.
2. The antigen binding molecule of paragraph 1, wherein the TRIM21 binding feature includes the interface of the CH2 and CH3 domains of an immunoglobulin constant region.
3. The antigen binding molecule of paragraphs 1 and 2, wherein the TRIM21 binding feature includes the CH2 and CH3 domains of an immunoglobulin constant region.
4. The antigen binding molecule of paragraphs 2 and 3, wherein the immunoglobulin is an IgG, optionally a human IgG, optionally an IgG1.
5. The antigen binding molecule of any one of paragraphs 1-4, wherein the cell-penetrating feature of a cell-penetrating antibody includes the cell-penetrating domain of an autoantibody.
6. The antigen binding molecule of any one of paragraphs 1-5, wherein the cell-penetrating feature of a cell-penetrating antibody includes the cell-penetrating domain of antibody 3E10 or 5C6, or variant or humanized form thereof.
7. The antigen binding molecule of any one of paragraphs 1-6, wherein the antigen binding feature that binds to a target protein is VH and VL domains, a single chain antibody, a single chain variable fragments (scFv), di-scFv, tri-scFv, diabody, triabody, teratbody, disulfide-linked Fvs (sdFv), Fab′, F(ab′)2, Fv, a single domain antibody fragments (sdAb), nanobody, or VHH.
8. The antigen binding molecule of any one of paragraphs 1-7, further including the complementary determining regions (CDR), and optionally the heavy and light chain variable regions of the cell-penetrating antibody.
9. The antigen binding molecule of any one of paragraphs 1-8, further including the sequence of TRIM21, or a functional fragment or variant thereof.
10. The antigen binding molecule of any one of paragraphs 1-9, wherein the molecule includes two or more an antigen binding features that bind different epitopes on the same protein.
11. The antigen binding molecule of any one of paragraphs 1-10, wherein the molecule includes two or more an antigen binding features that bind epitopes on different proteins.
12. The antigen binding molecule of any one of paragraphs 1-11, wherein the molecule is a multispecific antibody.
13. The antigen binding molecule of paragraph 12, wherein the molecule is a bi- or tri-specific antibody.
14. The antigen binding molecule of any one of paragraphs 1-11, wherein the molecule is a bispecific and trispecific IgG, IgG-scFv, IgG-dAb, scFv-Fc-scFv, knob-in-hole (KIH)-IgG, KA-body, KIH0Fc-Fab/scFv, tandom scFv, KIH trispecific, bispecific Fc fusion (N- or C-terminal, with or without KIH).
15. The antigen binding molecule of any one of paragraphs 1-14, including the six CDRs, and optionally the heavy and light chain variable regions of the 3E10 or 5C6 optionally selected from the CDRs and/or heavy and light chain variable regions provided herein, optionally the heavy and light chain variable regions of SEQ ID NOS: 12 and 14 respectively, or variant or humanized form thereof optionally with at least 70% sequence identity thereto,
an antigen binding domain for a target protein, and
an Fc domain.
16. A composition including an effective amount of the antigen binding molecule of any one of paragraphs 1-15.
17. The composition of paragraph 16 including any effective amount of two or more different antigen binding molecules.
18. The composition of paragraph 17, wherein two or more different antigen binding molecules target the same protein.
19. The composition of paragraphs 17 or 18, wherein two or more different antigen binding molecules target different proteins.
20. A method of targeted degradation of one or more target proteins including contacting cells with the composition of any one of paragraphs 16-19.
21. The method of paragraph 20, wherein the contacting occurs in vivo in a subject in need thereof.
22. The method of paragraph 21, wherein the composition is administered by injection or infusion to a subject in need thereof.
23. The method of paragraphs 21 or 22, wherein the subject has a disease or disorder.
24. The method of paragraph 23, wherein the disease or disorder is cause by overexpression and/or aberrant expression of the target protein and/or expression of a mutant thereof.
25. The method of paragraph 24, wherein the composition is administered in an effective amount to treat the disease or disorder.

### EXAMPLES

### Example 1: 3E10 Treatment of Cells Mediates Intracellular Degradation of RAD51

### Materials and Methods

HCC38, YUGASP, and U2OS cells were seeded into 6 well plates and treated with noted concentrations of 3E10 antibody (heavy and light chains of SEQ ID NOS: 12 and 14, respectively) for 3 days. After treatment, cells were trypsinized, pelleted, and protein extracted. Levels of RAD51 were quantified by western blot.

### Results

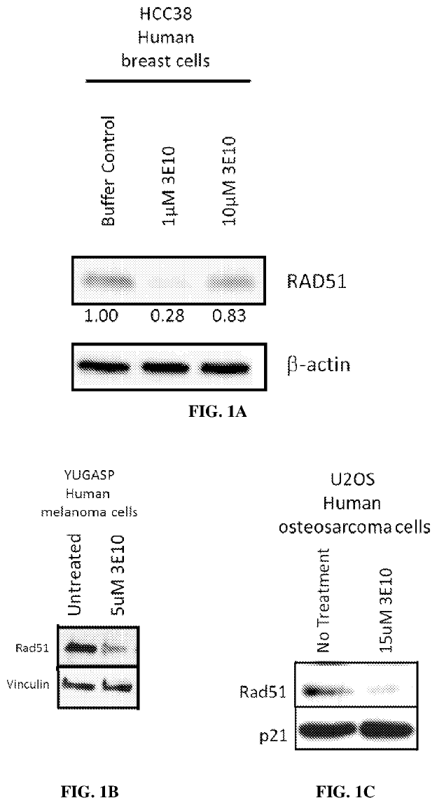

A modified version of the cell-penetrating antibody 3E10 mediates intracellular degradation of RAD51, for which 3E10 has moderate intrinsic binding affinity. See FIGS. 1A-1C. This is believed to be due to a pathway for degradation of intracellular antibodies that are bound to pathogens (and get internalized by cells in antibody/pathogen complexes) by the TRIM21 protein, which has ubiquitination activity. TRIM21 binds intracellular antibodies because it has a domain that binds to IgG Fc domains. Upon recruitment of multiple TRIM21 molecules, RING (SEQ ID NO: 127) domains become ubiquinated and are subsequently degraded along with the antibody-bound protein. As RAD51 typically exists in filaments or clusters, binding of full-length mAb 3E10 to RAD51 facilitated intracellular degradation through a ubiquitination pathway mediated by TRIM21.

### Example 2: Cell-Penetrating Bispecific Antibodies that Degrade Target Proteins of Interest

A bispecific antibody was designed where one Fab arm is 3E10 and another arm targets an intracellular antigen of interest (FIG. 2). These were generated using a standard CrossMab format adopting sequences from a parental 3E10 (heavy and light chains of SEQ ID NOS: 118 and 14, respectively) and an mAb targeting GFP (heavy and light chains of SEQ ID NO: 119 and 120, respectively). See also, Anti-GFP [N86/38.1R], Addgene plasmid #114492; Andrews, et al., Elife. 2019 Jan. 22; 8. pii: 43322. doi: 10.7554/eLife.43322. 10.7554/eLife.43322; Krah, “Engineering IgG-Like Bispecific Antibodies—An Overview” Antibodies 7, no. 3:28. doi.org/10.3390/antib7030028 (2018), and Klein, mAbs, 8:6, 1010-1020, DOI: 10.1080/19420862.2016.1197457 (2016).

>Heavy chain (3E10)
(SEQ ID NO: 118)
MGWSCIILFLVATATGVHSEVQLVESGGGLVKPGGSRKLSCAASGFTFSDY
GMHWVRQAPEKGLEWVAYISSGSSTIYYADTVKGRFTISRDNAKNTLFLQM
TSLRSEDTAMYYCARRGLLLDYWGQGTTLTVSAASTKGPSVFPLAPSSKST
SGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKA
KGQPREPQVYTLPPSRDELTKNQVSLWCLVKGFYPSDIAVEWESNGQPENN
YKTTPPVLDSDGSFFLYSKLTVDKSRWQQGNVFSCSVMHEALHNHYTQKSL
SLSPGK**
>Light chain (3E10)
(SEQ ID NO: 14)
MGWSCIILFLVATATGVHSDIVLTQSPASLAVSLGQRATISCRASKSVSTS
SYSYMHWYQQKPGQPPKLLIKYASYLESGVPARFSGSGSGTDFTLNIHPVE
EEDAATYYCQHSREFPWTFGGGTKLEIKRTVAAPSVFIFPPSDEQLKSGTA
SVVCLLNNFYPREAKVQWKVDNALQSGNSQESVTEQDSKDSTYSLSSTLTL
SKADYEKHKVYACEVTHQGLSSPVTKSFNRGEC**
>Heavy chain Crossed (GFP)
(SEQ ID NO: 119)
MGWSCIILFLVATATGVHSEVQLQQSGPELVKPGSSMKISCKASGYSFTGY
TMNWVKQSHGQNLEWIGLINPYNGGTNYNQKFKGKATLTVDKSSSTAYMEL
LGLTSEDSAVYYCTRGNSDFSAWFAYWGQGTSVTVSSASVAAPSVFIFPPS
DEQLKSGTASVVCLLNNFYPREAKVQWKVDNALQSGNSQESVTEQDSKDST
YSLSSTLTLSKADYEKHKVYACEVTHQGLSSPVTKSFNRGECDKTHTCPPC
PAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAP
IEKTISKAKGQPREPQVYTLPPSRDELTKNQVSLSCAVKGFYPSDIAVEWE
SNGQPENNYKTTPPVLDSDGSFFLVSKLTVDKSRWQQGNVFSCSVLHEALH
NRFTQKSLSLSPGK**
>Light chain Crossed (GFP)
(SEQ ID NO: 120)
MGWSCIILFLVATATGVHSDIQLTQSPAIMSPSLGERVTMTCTASSSVGSS
YLHWFQQKPGSSPKLWIYSTSNLASGVPARFSGSGSGTSYSLTISRMEAED
AATYYCHQYHRTPYTFGGGTKLEIKSSASTKGPSVFPLAPSSKSTSGGTAA
LGCLVKDYFPEPVTVSWNSGALTSGVHTFPAVLQSSGLYSLSSVVTVPSSS
LGTQTYICNVNHKPSNTKVDKKVEPKSC**

### Example 3: Cell-Penetrating Bispecific Antibodies are Rapidly Degraded by the Proteosome

### Materials and Methods

B16 cells in culture were pretreated with and without proteasomal inhibitor MG132. Following 60 minutes of treatment, cells were treated with 1 μM of 3E10/GFP bispecific mAb of Example 2. After 24 hours of treatment, cells were harvested and the levels of bispecific antibodies were monitored by Western blot.

### Results

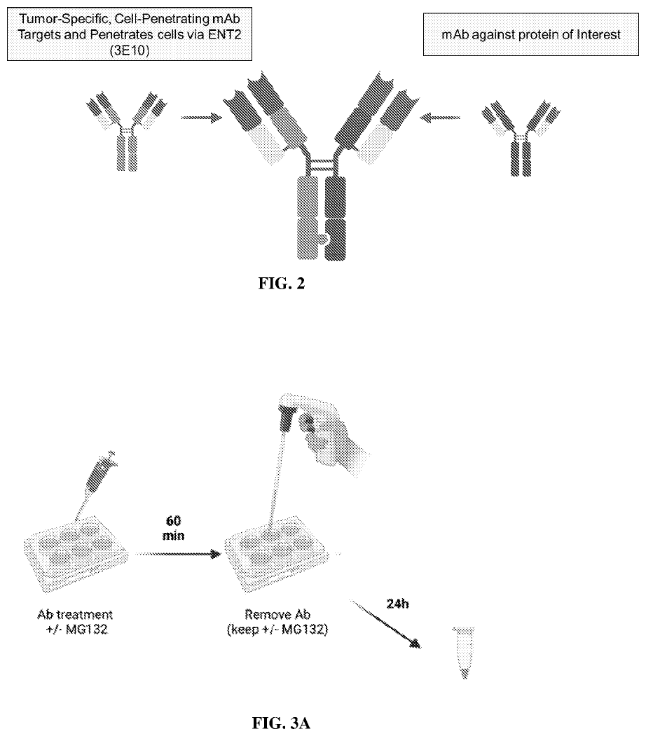

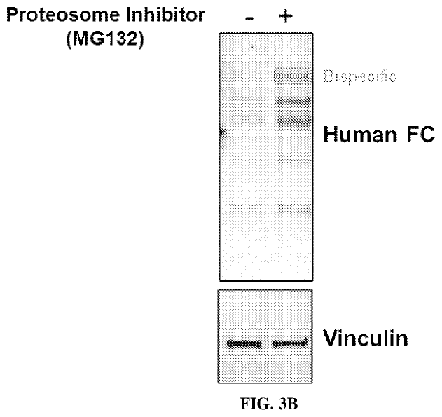

FIGS. 3A-3B show and assay (FIG. 3A) and its results (FIG. 3B) for intracellular degradation of the bispecific antibody mediated by the proteosome. The use of proteosome inhibitor, MG132, rescues the signal as illustrated by the western blot in the FIG. 3B.

### Example 4: Cell-Penetrating Bispecific Antibodies Mediate Nuclear Protein Degradation of Target Proteins

### Materials and Methods

293T cells in culture were transfected with GFP-NLS mRNA. Cells were treated with the 3E10/GFP bispecific antibody of Example 2 twenty-four hours later, and analyzed by immunofluorescence imaging and flow cytometry. One Fab arm is 3E10 and another arm targets GFP.

### Results

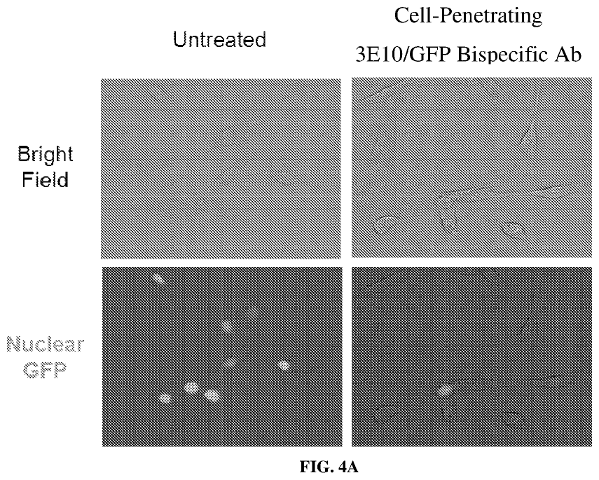

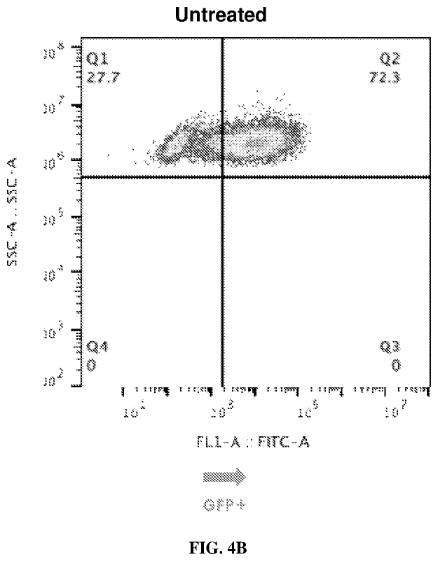

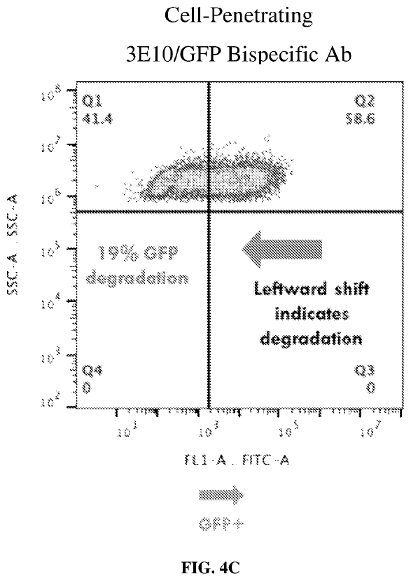

Experiments were designed to determine if the 3E10/GFP bispecific antibody increases degradation of target proteins. Cells were treated with the bispecific antibody of Example 1. Results show that introducing bispecific antibodies to 293T cells leads to decreased fluorescence after 24 hours indicating degradation of nuclear green fluorescent protein (GFP) levels as demonstrated by immunofluorescence images in the FIG. 4A. Results obtained using flow cytometry show 19% degradation in GFP levels (FIGS. 4B and 4C) indicating targeted degradation of GFP using this bispecific antibody compared to untreated control.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

We claim:

1. An antigen binding molecule comprising (i) a TRIM21 binding feature, (ii) the cell-penetrating feature of a cell-penetrating antibody, and (iii) an antigen binding feature that binds to a target protein.

2. The antigen binding molecule of claim 1, wherein the TRIM21 binding feature comprises the interface of the CH2 and CH3 domains of an immunoglobulin constant region.

3. The antigen binding molecule of claims 1 and 2, wherein the TRIM21 binding feature comprises the CH2 and CH3 domains of an immunoglobulin constant region.

4. The antigen binding molecule of claims 2 and 3, wherein the immunoglobulin is an IgG, optionally a human IgG, optionally an IgG1.

5. The antigen binding molecule of any one of claims 1-4, wherein the cell-penetrating feature of a cell-penetrating antibody comprises the cell-penetrating domain of an autoantibody.

6. The antigen binding molecule of any one of claims 1-5, wherein the cell-penetrating feature of a cell-penetrating antibody comprises the cell-penetrating domain of antibody 3E10 or 5C6, or variant or humanized form thereof.

7. The antigen binding molecule of any one of claims 1-6, wherein the antigen binding feature that binds to a target protein is VH and VL domains, a single chain antibody, a single chain variable fragments (scFv), di-scFv, tri-scFv, diabody, triabody, teratbody, disulfide-linked Fvs (sdFv), Fab′, F(ab′)2, Fv, a single domain antibody fragments (sdAb), nanobody, or VHH.

8. The antigen binding molecule of any one of claims 1-7, further comprising the complementary determining regions (CDR), and optionally the heavy and light chain variable regions of the cell-penetrating antibody.

9. The antigen binding molecule of any one of claims 1-8, further comprising the sequence of TRIM21, or a functional fragment or variant thereof.

10. The antigen binding molecule of any one of claims 1-9, wherein the molecule comprises two or more an antigen binding features that bind different epitopes on the same protein.

11. The antigen binding molecule of any one of claims 1-10, wherein the molecule comprises two or more an antigen binding features that bind epitopes on different proteins.

12. The antigen binding molecule of any one of claims 1-11, wherein the molecule is a multispecific antibody.

13. The antigen binding molecule of claim 12, wherein the molecule is a bi- or tri-specific antibody.

14. The antigen binding molecule of any one of claims 1-11, wherein the molecule is a bispecific and trispecific IgG, IgG-scFv, IgG-dAb, scFv-Fc-scFv, knob-in-hole (KIH)-IgG, κλ-body, KIH0Fc-Fab/scFv, tandom scFv, KIH trispecific, bispecific Fc fusion (N- or C-terminal, with or without KIH).

15. The antigen binding molecule of any one of claims 1-14, comprising the six CDRs, and optionally the heavy and light chain variable regions of the 3E10 or 5C6 optionally selected from the CDRs and/or heavy and light chain variable regions provided herein, optionally the heavy and light chain variable regions of SEQ ID NOS: 12 and 14 respectively, or variant or humanized form thereof optionally with at least 70% sequence identity thereto,
an antigen binding domain for a target protein, and
an Fc domain.

16. A composition comprising an effective amount of the antigen binding molecule of any one of claims 1-15.

17. The composition of claim 16 comprising any effective amount of two or more different antigen binding molecules.

18. The composition of claim 17, wherein two or more different antigen binding molecules target the same protein.

19. The composition of claim 17 or 18, wherein two or more different antigen binding molecules target different proteins.

20. A method of targeted degradation of one or more target proteins comprising contacting cells with the composition of any one of claims 16-19.

21. The method of claim 20, wherein the contacting occurs in vivo in a subject in need thereof.

22. The method of claim 21, wherein the composition is administered by injection or infusion to a subject in need thereof.

23. The method of claim 21 or 22, wherein the subject has a disease or disorder.

24. The method of claim 23, wherein the disease or disorder is cause by overexpression and/or aberrant expression of the target protein and/or expression of a mutant thereof.

25. The method of claim 24, wherein the composition is administered in an effective amount to treat the disease or disorder.


SEQUENCE LISTING (128 sequences)

SEQ ID NO: 1 - protein, 116 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 2 - protein, 116 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 116 aa, synthetic construct
EVQLVQSGGGLIQPGGSLRLSCAASGFTFSNYGMHWVRQAPGKGLEWVSYISSGSSTIYYADSVKGRFTISRDNSKNTLYLQMNSLRAEDTAVYYCARRGLLLDYWGQGTTVTVSS

SEQ ID NO: 4 - protein, 116 aa, synthetic construct
EVQLVESGGGLIQPGGSLRLSCAASGFTFSNYGMHWVRQAPGKGLEWVSYISSGSSTIYYADSVKGRFTISRDNSKNTLYLQMTSLRAEDTAVYYCARRGLLLDYWGQGTTLTVSS

SEQ ID NO: 5 - protein, 116 aa, synthetic construct
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 6 - protein, 116 aa, synthetic construct
EVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 7 - protein, 111 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 8 - protein, 111 aa, synthetic construct
DIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELK

SEQ ID NO: 9 - protein, 111 aa, synthetic construct
DIQMTQSPSSLSASVGDRVTITCRASKSVSTSSYSYLAWYQQKPEKAPKLLIKYASYLQSGVPSRFSGSGSGTDFTLTISSLQPEDFATYYCQHSREFPWTFGAGTKLELK

SEQ ID NO: 10 - protein, 111 aa, synthetic construct
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 11 - protein, 111 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIK

SEQ ID NO: 12 - protein, 465 aa, synthetic construct
MGWSCIILFLVATATGVHSEVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 465 aa, synthetic construct
MGWSCIILFLVATATGVHSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 237 aa, synthetic construct
MGWSCIILFLVATATGVHSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 5 aa, synthetic construct
DYGMH

SEQ ID NO: 16 - protein, 5 aa, synthetic construct
NYGMH

SEQ ID NO: 17 - protein, 17 aa, synthetic construct
YISSGSSTIYYADTVKG

SEQ ID NO: 18 - protein, 7 aa, synthetic construct
RGLLLDY

SEQ ID NO: 19 - protein, 17 aa, synthetic construct
YISSGSSTIYYADSVKG

SEQ ID NO: 20 - protein, 8 aa, synthetic construct
GFTFSDYG

SEQ ID NO: 21 - protein, 8 aa, synthetic construct
GFTFSNYG

SEQ ID NO: 22 - protein, 8 aa, synthetic construct
ISSGSSTI

SEQ ID NO: 23 - protein, 9 aa, synthetic construct
ARRGLLLDY

SEQ ID NO: 24 - protein, 15 aa, synthetic construct
RASKSVSTSSYSYMH

SEQ ID NO: 25 - protein, 7 aa, synthetic construct
YASYLES

SEQ ID NO: 26 - protein, 9 aa, synthetic construct
QHSREFPWT

SEQ ID NO: 27 - protein, 15 aa, synthetic construct
RASKSVSTSSYSYLA

SEQ ID NO: 28 - protein, 7 aa, synthetic construct
YASYLQS

SEQ ID NO: 29 - protein, 10 aa, synthetic construct
KSVSTSSYSY

SEQ ID NO: 30 - skipped

SEQ ID NO: 31 - protein, 7 aa, synthetic construct
GQSSRSS

SEQ ID NO: 32 - protein, 18 aa, synthetic construct
GQSSRSSSGGGSSGGGGS

SEQ ID NO: 33 - protein, 4 aa, synthetic construct
GSGS

SEQ ID NO: 34 - protein, 4 aa, synthetic construct
GGGS

SEQ ID NO: 35 - protein, 10 aa, synthetic construct
GGGGSGGGGS

SEQ ID NO: 36 - protein, 20 aa, synthetic construct
GGGGSGGGGSGGGGSGGGGS

SEQ ID NO: 37 - protein, 15 aa, synthetic construct
GGGGSGGGGSGGGGS

SEQ ID NO: 38 - protein, 274 aa, synthetic construct
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 39 - protein, 541 aa, synthetic construct
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 40 - protein, 808 aa, synthetic construct
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 41 - protein, 242 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIKGGGGSGGGGSGGGGSEVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 42 - protein, 17 aa, synthetic construct
YISSSSSTIYYADSVKG

SEQ ID NO: 43 - protein, 8 aa, synthetic construct
ISSSSSTI

SEQ ID NO: 44 - protein, 15 aa, synthetic construct
RASKTVSTSSYSYMH

SEQ ID NO: 45 - protein, 10 aa, synthetic construct
KTVSTSSYSY

SEQ ID NO: 46 - protein, 116 aa, synthetic construct
EVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 47 - protein, 116 aa, synthetic construct
EVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 48 - protein, 116 aa, synthetic construct
EVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 49 - skipped

SEQ ID NO: 50 - protein, 116 aa, synthetic construct
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 51 - protein, 116 aa, synthetic construct
EVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 52 - protein, 116 aa, synthetic construct
EVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 53 - protein, 111 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIK

SEQ ID NO: 54 - protein, 111 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIK

SEQ ID NO: 55 - protein, 111 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIK

SEQ ID NO: 56 - protein, 111 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 57 - protein, 111 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIK

SEQ ID NO: 58 - protein, 111 aa, synthetic construct
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIK

SEQ ID NO: 59 - protein, 21 aa, synthetic construct
RADAAPGGGGSGGGGSGGGGS

SEQ ID NO: 60 - protein, 19 aa, synthetic construct
ASTKGPSVFPLAPLESSGS

SEQ ID NO: 61 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 62 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 63 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 64 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 65 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 66 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 67 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 68 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 69 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 70 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 71 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 72 - protein, 515 aa, unidentified
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 73 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 74 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 75 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 76 - protein, 515 aa, synthetic construct
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 77 - protein, 446 aa, synthetic construct
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 79 - protein, 12 aa, synthetic construct
EPKSCDKTHTCP

SEQ ID NO: 80 - protein, 113 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 81 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 446 aa, synthetic construct
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 113 aa, synthetic construct
PCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAK

SEQ ID NO: 84 - protein, 446 aa, synthetic construct
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 113 aa, synthetic construct
PCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAK

SEQ ID NO: 86 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 87 - protein, 218 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 31 aa, synthetic construct
RASKSVSTSSYSYMHWYQQKPGQPPKLLIKY

SEQ ID NO: 89 - protein, 31 aa, synthetic construct
RASKTVSTSSYSYMHWYQQKPGQPPKLLIKY

SEQ ID NO: 90 - protein, 33 aa, synthetic construct
RVTITCRASKSVSTSSYSYMHWYQQKPGKAPKL

SEQ ID NO: 91 - protein, 32 aa, synthetic construct
XRASKTVSTSSYSYMHWYQQKPGQPPKLLXKY

SEQ ID NO: 92 - protein, 115 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVS

SEQ ID NO: 93 - protein, 132 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGS

SEQ ID NO: 94 - protein, 112 aa, synthetic construct
DIVLTQSPASLAAVSLGERATISYRASKSVSTSGYSYMHWNQQKPGQAPRLLIYLVSNLESGVPARFSGSGSGTDFTLNIHPVEEEDAATYYCQHIRELDTFFGGGTKLEIK

SEQ ID NO: 95 - protein, 15 aa, synthetic construct
RASKSVSTSGYSYMH

SEQ ID NO: 96 - protein, 7 aa, synthetic construct
LVSNLES

SEQ ID NO: 97 - protein, 9 aa, synthetic construct
QHIRELDTF

SEQ ID NO: 98 - protein, 120 aa, synthetic construct
QLKLVESGGGLVKPGGSLKLSCAASGFTFSSYTMSWVRQTPAKRLEWVATISSGGGSTYYPDSVKGRFTISRDNARNTLYLQMSSLRSEDTAMYYCARRAYSKRGAMDYWGQGTSVTVSS

SEQ ID NO: 99 - protein, 5 aa, synthetic construct
SYTMS

SEQ ID NO: 100 - protein, 17 aa, synthetic construct
TISSGGGSTYYPDSVKG

SEQ ID NO: 101 - protein, 11 aa, synthetic construct
RAYSKRGAMDY

SEQ ID NO: 102 - protein, 475 aa, Homo sapiens [sequence omitted]

SEQ ID NO: 103 - protein, 465 aa, synthetic construct
MGWSCIILFLVATATGVHSEVQLVESGGGLVKPGGSRKLSCAASGFTFSRYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 465 aa, synthetic construct
MGWSCIILFLVATATGVHSEVQLVESGGGLVKPGGSRKLSCAASGFTFSKYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 237 aa, synthetic construct
MGWSCIILFLVATATGVHSDIVLTQSPASLAVSLGQRATISCRASKSVDTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 237 aa, synthetic construct
MGWSCIILFLVATATGVHSDIVLTQSPASLAVSLGQRATISCRASKSVSTSSYSYMHWYQQKPGQPPKLLIKYASYLESGVPARFSGSGSGTDFTLNIHPVEEEDAATYYCQHSNEFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 5 aa, synthetic construct
RYGMH

SEQ ID NO: 108 - protein, 5 aa, synthetic construct
KYGMH

SEQ ID NO: 109 - protein, 8 aa, synthetic construct
GFTFSRYG

SEQ ID NO: 110 - protein, 8 aa, synthetic construct
GFTFSKYG

SEQ ID NO: 111 - protein, 115 aa, synthetic construct
EVQLVESGGGLVKPGGSRKLSCAASGFTFSRYGMHWVRQAPEKGLEWVAYISSGSSTIYYADTVKGRFTISRDNAKNTLFLQMTSLRSEDTAMYYCARRGLLLDYWGQGTTLTVS

SEQ ID NO: 112 - protein, 115 aa, synthetic construct
EVQLVESGGGLVKPGGSRKLSCAASGFTFSKYGMHWVRQAPEKGLEWVAYISSGSSTIYYADTVKGRFTISRDNAKNTLFLQMTSLRSEDTAMYYCARRGLLLDYWGQGTTLTVS

SEQ ID NO: 113 - protein, 15 aa, synthetic construct
RASKSVDTSSYSYMH

SEQ ID NO: 114 - protein, 9 aa, synthetic construct
QHSNEFPWT

SEQ ID NO: 115 - protein, 10 aa, synthetic construct
KSVDTSSYSY

SEQ ID NO: 116 - protein, 112 aa, synthetic construct
DIVLTQSPASLAVSLGQRATISCRASKSVDTSSYSYMHWYQQKPGQPPKLLIKYASYLESGVPARFSGSGSGTDFTLNIHPVEEEDAATYYCQHSREFPWTFGGGTKLEIKR

SEQ ID NO: 117 - protein, 111 aa, synthetic construct
DIVLTQSPASLAVSLGQRATISCRASKSVDTSSYSYMHWYQQKPGQPPKLLIKYASYLESGVPARFSGSGSGTDFTLNIHPVEEEDAATYYCQHSREFPWTFGGGTKLEIK

SEQ ID NO: 118 - protein, 465 aa, synthetic construct
MGWSCIILFLVATATGVHSEVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 473 aa, synthetic construct
MGWSCIILFLVATATGVHSEVQLQQSGPELVKPGSSMKISCKASGYSFTGYTMNWVKQSHGQNLEWIGLINPYNGGTNYNQKFKGKATLTVDKSSSTAYMELLGLTSEDSAVYYCTRGNSDFSAWFAYWGQGTSVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHNRFTQKSLSLSPGK

SEQ ID NO: 120 - protein, 232 aa, synthetic construct
MGWSCIILFLVATATGVHSDIQLTQSPAIMSPSLGERVTMTCTASSSVGSSYLHWFQQKPGSSPKLWIYSTSNLASGVPARFSGSGSGTSYSLTISRMEAEDAATYYCHQYHRTPYTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 121 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 334 aa, synthetic construct
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHNRFTQKSLSLSPGK

SEQ ID NO: 123 - protein, 107 aa, synthetic construct [sequence omitted]

SEQ ID NO: 124 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 125 - protein, 7 aa, synthetic construct
PRYSPRY

SEQ ID NO: 126 - protein, 4 aa, synthetic construct
SPRY

SEQ ID NO: 127 - protein, 4 aa, synthetic construct
RING

SEQ ID NO: 128 - protein, 112 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSNEFPWTFGGGTKLEIKR